(19) United States
(12) Patent Application Publication
(10) Pub. No.: US 2025/0345797 A1
(43) Pub. Date: Nov. 13, 2025

(54) MICROFLUIDIC DEVICES AND METHODS FOR SORTING PARTICLES SUCH AS EXTRACELLULAR VESICLES IN A SAMPLE

(71) Applicant: The Hong Kong University of Science and Technology, Hong Kong (CN)

(72) Inventors: Levent YOBAS, Hong Kong (CN); Yang BU, Hong Kong (CN)

(21) Appl. No.: 19/201,968

(22) Filed: May 8, 2025

Related U.S. Application Data
(60) Provisional application No. 63/643,937, filed on May 8, 2024.

(51) Int. Cl. B01L 3/00; C12N 5/078

(52) U.S. Cl. CPC B01L 3/502761; C12N 5/0634; B01L 2200/0652; B01L 2200/16; B01L 2300/0864; B01L 2400/0418

(57) ABSTRACT

In some embodiments, provided is microfluidic devices and methods for sorting particles in a sample. In some embodiments, the microfluidic device comprises a sample inlet for sample loading; at least one reservoir; a sieving array; and at least one outlet for collecting any sorted particles. Other example embodiments are described herein. In certain embodiments, the microfluidic devices and methods provide simple, rapid, efficient and versatile solutions for sorting particles such as extracellular vesicles.

### CROSS-REFERENCE TO RELATED APPLICATION

This application claims benefit under 35 U.S.C. § 119 (e) of U.S. Provisional Application having Ser. No. 63/643,937 filed May 8, 2024, the entire contents of which is/are hereby incorporated by reference herein.

### FIELD OF INVENTION

This application relates to microfluidics, in particular, microfluidic devices and methods of sorting particles such as extracellular vesicles.

### BACKGROUND OF THE INVENTION

Extracellular vesicles (EVs) are membrane-enclosed transport carriers secreted by cells that carry a variety of biomolecules such as nucleic acids and proteins, inheriting the biological signature of the parent cells and serving as mediators of intercellular communications. Cargos carried by EVs can reflect disease status and serve as crucial biomarkers for early disease diagnosis, thus exhibiting significant potential in liquid biopsies. EVs can be classified by their size into small EVs (<200 nm), which contain exosomes (30-150 nm), and large EVs (>200 nm), which are mainly composed of microvesicles and apoptotic bodies. Developing size fractionation methods for EV subpopulations is crucial because EVs have been proven to contain different molecular components depending on their size and origin, which have significant clinical implications. To date, the main challenge in EV size fractionation comes from their small size range, and difficulty in complete removal of the abundant contaminants, including free nucleic acids and proteins, which interfere with downstream analysis and clinical diagnosis.

The conventional approaches have been developed in the past decades aiming at isolation and purification of specific EV subpopulations, but all have certain limitations despite their merits. Density gradient ultracentrifugation, the main and gold standard technique for EV isolation, suffers from low EV purity and integrity, as well as lengthy processing times, which limit its clinical applicability. Precipitation-based methods, such as polyethylene glycol (PEG)-based precipitation, can isolate EVs from large volumes of samples in a single step, but they have drawbacks such as polymer interference, low EV purity and inability to target specific EV subpopulations. Size-based microfiltration and ultrafiltration can isolate EV subgroups using membranes with well-defined pore sizes, but they also co-extract proteins and apply high shear stress that can potentially damage EVs. Immunoaffinity methods label and isolate EVs that have specific surface proteins with antibodies, but the capture efficiency is influenced by the heterogeneous expression levels of antigens on EVs. Agarose gel electrophoresis is able to separate EVs from various lipoproteins based on their electrophoretic (EP) mobility differences. However, the gel electrophoresis platform cannot isolate EV subpopulations and requires>3 hours to resolve the fraction bands and recover the isolated sample from the excised gel segment.

In recent years, microfluidic approaches have been attempted to isolate particles such as EV subpopulations and principles used by these devices include surface acoustic waves, asymmetrical flow field flow fractionation, deterministic lateral displacement, ultrafiltration, viscoelastic flow, dielectrophoresis, and electrophoresis. Nevertheless, the current microfluidic platforms still need further development to meet the high standards for clinical applications, such as user-friendly operation without specialized equipment, better resolution to isolate particles such as EVs with small size differences, capability to remove nanoscale contaminants, and additive molecules with enhanced efficiency to shorten separation procedure. A few groups reported separation of EVs and their subpopulations by performing electrophoresis inside capillaries (capillary electrophoresis). These conventional approaches require long capillaries (>30 cm) and are for analytical purposes, by on-site detection of EV subgroups, and have drawbacks such as requiring laboratory settings and high voltage supply (usually >10 kV), lengthy sample processing and collection procedure. Several other groups utilize on-chip capillary electrophoresis for the analysis of EVs but only to determine the EP values of the EVs from various cell origins. Therefore, there is a demand to further advance the microfluidic platforms for isolation of particles such as EV subpopulations.

### BRIEF SUMMARY OF THE INVENTION

Disclosed herein are novel devices, kits, methods and uses that are useful for sorting particles such as EVs, processes for preparing the devices, methods of using the devices, and intermediates used in preparing the devices.

In one aspect, provided herein is a microfluidic device for sorting particles in a sample, including: a sample inlet for sample loading; at least one reservoir; a sieving array; and at least one outlet for collecting any sorted particles, wherein the sample inlet, the at least one reservoir and the at least one particle collection reservoir are in fluid communication with the sieving array, respectively, wherein the sieving array includes a substrate and a plurality of pillars, the plurality of pillars are spaced from one another and arranged in an array of a plurality of rows substantially in x direction and a plurality of columns substantially in y direction, wherein each two adjacent columns define a well therebetween, thereby forming a plurality of wells in the sieving array, wherein each two adjacent pillars in the same column further define a slit therebetween, thereby forming a plurality of slits in the sieving array, such that, when in operation, particles are driven to pass through one or more of the plurality of slits and/or one or more of the plurality of wells based on at least particle size, thereby particles are sorted and collected from the at least one outlet.

In another aspect, provided herein is a microfluidic device for size fractionation, separation or purification of extracellular vesicles (EVs) in a sample, including: at least one sample inlet connected with an injection channel for sample loading; a plurality of buffer reservoirs; a sieving array; and a plurality of outlets connected with sample collection reservoirs for collecting sorted EVs, wherein the sample inlet, the at least one reservoir and the at least one particle collection reservoir are in fluid communication with the sieving array via one or more microchannels, respectively, wherein the sieving array includes a substrate and a plurality of pillars, the plurality of pillars are spaced from one another and arranged in an array of a plurality of rows substantially in x direction and a plurality of columns substantially in y direction, wherein each two adjacent columns define a well therebetween, thereby forming a plurality of wells in the sieving array, wherein each two adjacent pillars in the same column further define a slit therebetween, thereby forming a plurality of slits in the sieving array, wherein each well has a well depth and a well width, and each slit has a slit depth and a slit length; and wherein the slit depth is configured to be smaller than the well depth; and/or the slit length is configured to be smaller than the well width, such that, when in operation, EVs are driven to pass through one or more of the plurality of slits and/or one or more of the plurality of wells based on at least particle size, thereby EVs are sorted and collected in the plurality of outlets.

In another aspect, provided herein is a method for sorting particles in a sample, comprising the steps of: (a) providing a device as described in any one of the embodiments; (b) loading a sample to the sample inlet of the device; and (c) flowing the sample into the device, such that particles are sorted and collected in the at least one outlet.

In another aspect, provided herein is a method for size fractionation, separation or purification of extracellular vesicles (EVs) in a sample, comprising the steps of: (a) providing a device as described in any one of the embodiments; (b) loading a sample to the sample inlet of the device; and (c) flowing the sample into the device, such that EVs are sorted and collected in the at least one outlet.

There are many advantages of the invention. In certain embodiments, the microfluidic devices and methods provide simple, rapid, efficient and versatile solutions for sorting particles such as extracellular vesicles.

### BRIEF DESCRIPTION OF THE DRAWINGS

FIG. 1A shows a schematic diagram of the 2D EP size fractionation of EVs in a microfluidic artificial sieve device. The artificial sieve device fractionates EV subpopulations based on their sizes, which shows larger vesicles deflect more than the smaller ones, and simultaneously purifies them from soluble proteins and short nucleic acids. The fractionated EVs are directed into designated outlet ports for collection. FIG. 1B has a left panel showing SEM images of a representative artificial sieve before bonding the PDMS cover slab in top view and side view, which comprises slits, wells and pillars. Schematic diagram superimposed on SEM image describes the example trajectories of small and large particles across the artificial sieve, according to an example embodiment. Moreover, FIG. 1B has a right panel showing the structures with a sectioned schematic view indicating large and small particles migrating across the microslit-well sieving array structure under anisotropic electric field, according to an embodiment of the subject invention. FIG. 1C is a schematic diagram showing an example microfluidic device according to an example embodiment. FIG. 1D shows an example microfluidic device layout according to an example embodiment, and voltage supply settings for size fractionation of particles such as EV subpopulations. The center region of the sieving array (grey-colored square area, 130′) has a dimension of 5 mm×5 mm. The sieving array is connected to the surrounding reservoirs through an array of fluidic microchannels. Each microchannel imposes a high electrical resistance and thus serves as a current-injection source to establish tunable uniform electric fields across the sieving array, according to an embodiment of the subject invention. FIG. 1E is a schematic diagram showing the structures of an example sieving array, according to an example embodiment. FIG. 1F is a sectioned schematic view of the structures of an example sieving array, according to the same example embodiment of FIG. 1E. FIG. 1G is a schematic diagram showing the structures of an example sieving array, according to another example embodiment. FIG. 1H is a sectioned schematic view of the structures of an example sieving array, according to the same example embodiment of FIG. 1G. FIG. 1H′ is another sectioned schematic view of the structures of another example sieving array, according to the same example embodiment of FIG. 1G.

FIG. 2A shows representative fluorescent images captured near the inlet showing the size separation of 300 nm and 70 nm fluorescent particles under varying Eₓ and E_{y}=60 V cm⁻¹. Injection channel width: 34 μm. FIG. 2B shows a high magnification image captured near the outlets of the artificial sieve device showing the fractionated streams of 300 nm and 70 nm particles, under Eₓ=20 V cm⁻¹ and E_{y}=60 V cm⁻¹. FIG. 2C shows a fluorescent image captured near the outlets, showing particle streams are deflected to designated collection ports, under Eₓ=50 V cm⁻¹ and E_{y}=60 V cm⁻¹. Collection outlet width: 1 mm for Outlet I and 4 mm for Outlet II. FIG. 2D shows superimposed histograms demonstrating size distributions of the particles collected from the corresponding outlets, analyzed via Nanoparticle Tracking Analysis (NTA). FIG. 2E shows a plot of tangent θ as a function of Eₓ under E_{y}=60 V cm 1, for 70 nm and 300 nm particles (fitting lines, a quadratic fit, n=3, R²>0.99). All experiments shown here are conducted with 0.5×tris-borate-EDTA (TBE) buffer, through the artificial sieve device with 1 μm deep slit and 4 μm deep well, 4 μm pitch. Scale bars: 200 μm, according to an embodiment of the subject invention.

FIG. 3 shows a fluorescent image captured near outlet channels showing the size fractionation of a ternary mixture of 70, 200 and 300 nm particles. Superimposed fluorescence intensity (in arbitrary units) profiles plotted along the exit ports (dashed line). Histograms showing NTA data of the particles collected from corresponding outlets show distinct separation. Collection outlet width: 0.45 mm for all the three outlets. Electric field: Eₓ=40 V cm⁻¹; E_{y}=60 V cm⁻¹; Working buffer: 0.5×TBE, according to an embodiment of the subject invention.

FIG. 4A shows a composite fluorescent image taken near the outlet channels of the artificial sieve device, demonstrating size fractionation of EVs (stained with PKH26 dye). Scale bar: 200 μm. FIG. 4B shows histograms demonstrating NTA analysis of the EVs collected from outlets. Insets: the negative staining electron microscopic images of the collected EVs with different size. Scale bars: 200 nm. FIG. 4C shows trajectories of a smaller EV (light) and a larger EV (dark) that reveal the size-dependent migration path and deflection angles. FIG. 4D shows correlation between optical cross-sectional area of single EVs (indicative of relative EV size) and their deflection angles, based on an analysis of high-magnification images. Electric field: Eₓ=40 V cm⁻¹, E)=60 V cm⁻¹; Working buffer: 0.5×TBE, according to an embodiment of the subject invention.

FIGS. 5A-5B show fluorescent images demonstrating distinct streams indicating separation of small EVs (sEV) from FIG. 5A illustrating protein contamination ovalbumin and FIG. 5B illustrating 500 bp DNA under electric field Eₓ=60 V cm⁻¹, E_{y}=60 V cm⁻¹. Working buffer: 0.5×TBE. Scale bars: 200 μm, according to an embodiment of the subject invention.

FIG. 6 shows device layout with microphotographs showing the detailed structures of the artificial sieve device of the subject invention, and voltage supply settings for size fractionation of EV subpopulations. The center region of the sieving array (grey-colored square area) has a dimension of 5 mm×5 mm. The sieving array is connected to the surrounding reservoirs through an array of fluidic microchannels (microphotographs). Each microchannel imposes a high electrical resistance and thus serves as a current-injection source to establish tunable uniform electric fields across the sieving array, according to an embodiment of the subject invention.

FIG. 7 has a left panel showing plot of the separation distance Δx between 70 and 300 nm particle streams as a function of the applied electric field Eₓ along x-axis (E, is fixed at 60 V cm⁻¹). Right panel shows corresponding fluorescent images taken near the outlet collection channels. Scale bars: 300 μm, according to an embodiment of the subject invention.

FIG. 8 shows a plot of the electrophoretic velocity of 300 nm particles migrating along the y direction through deep wells in relation to the electric field strength E_{y} applied (Eₓ=0). Data were obtained from tracking the motion of individual particles (n=5). Electrophoretic velocity is calculated by the measured migration length divided by respective migration time. The dashed line shows a quadratic fit (R²=0.99), according to an embodiment of the subject invention.

FIG. 9A shows configuration of isotropic micropillar array with 2 μm spacing, FIG. 9B shows a SEM image of the isotropic micropillar array with 2 μm spacing, FIG. 9C shows fluorescent images taken near the entrance of the artificial sieve device, and FIG. 9D shows fluorescent images taken near collection channels demonstrating unsuccessful separation of 70/300 nm particle mixture in the isotropic micropillar array. Eₓ=50 V cm⁻¹, E_{y}=60 V cm⁻¹, with scale bars of 200 μm, according to an embodiment of the subject invention.

FIG. 10 shows plots of deflection angle tangent θ as a function of Eₓ with E_{y}=60 V cm⁻¹ for 70 nm and 300 nm particles, wherein the experiments are conducted by the artificial sieve devices with slit depth 0.7 μm, 1 μm and 1.5 μm, with well depth 4 μm, with a working buffer of 0.5×TBE, according to an embodiment of the subject invention.

FIG. 11 shows the velocity of 70 nm particle stream under different TBE buffer conditions, with Eₓ=0 V cm⁻¹ and E_{y}=60 V cm⁻¹, according to an embodiment of the subject invention.

FIG. 12A shows superimposed fluorescent images captured near the collection channels showing separation of 120 nm (left region indicated by the dashed lines) and 70 nm (right indicated by dashed lines) particles. Vertical dashed lines are the guides to eyes, and FIG. 12B shows fluorescence intensity profiles plotted along horizontal dashed line near the exit port showing axial distribution of particle streams, according to an embodiment of the subject invention.

FIG. 13 shows plot diagrams of mean fluorescence intensity of the outlet reservoir over time, during a continuous-flow separation process under a low electric field strength of Eₓ=50 V cm⁻¹, E_{y}=60 V cm⁻¹ and high electric field strength of Eₓ=250 V cm⁻¹, E_{y}=300 V cm⁻¹, wherein the inset images show the collection of target EVs (stained) into outlet reservoir before and after 350 seconds of fractionation, according to an embodiment of the subject invention.

FIG. 14 has a left panel showing a schematic diagram of a microfluidic artificial sieve device, according to an embodiment of the subject invention. The artificial sieve device fractionates particles such as EV subpopulations based on their sizes, which shows larger vesicles deflect more than the smaller ones, and simultaneously purifies them from free proteins and short nucleic acids. FIG. 14 also has a right upper panel showing the migration of large and small particles from a well through a slit to another well with the artificial sieve, and the flow of the large and small particles in a free solution. FIG. 14 also has a right lower panel showing a composite fluorescent image taken near the outlet channels of the artificial sieve device, demonstrating size fractionation of EVs in different sizes (circulated in broken circles).

### DETAILED DISCLOSURE OF THE INVENTION

Embodiments of the subject invention are directed to microfluidic devices containing an artificial sieve, uses thereof, methods for fabricating the same, methods of sorting, separating, purifying or fractionating particles such as EVs.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. Where a range is referred in the specification, the range is understood to include each discrete point within the range. For example, 1-7 means 1, 2, 3, 4, 5, 6, and 7. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

As used herein, the term “about” is understood as within a range of normal tolerance in the art and can be ±10% of a stated value. By way of example only, about 50 means from 45 to 55 including all values in between. As used herein, the phrase “about” a specific value also includes the specific value, for example, about 50 includes 50.

As used herein and in the claims, the terms “general” or “generally”, or “substantial” or “substantially” mean that the recited characteristic, angle, shape, state, structure, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, an object that has a “generally” cylindrical shape would mean that the object has either an exact cylindrical shape or a nearly exact cylindrical shape. In another example, an object that is “substantially” perpendicular to a surface would mean that the object is either exactly perpendicular to the surface or nearly exactly perpendicular to the surface, e.g., has a 5% deviation.

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

For the sake of clarity, “comprising”, including, and “containing”, and any related forms are open-ended terms which allows for additional elements or features beyond the named essential elements, whereas “consisting of” is a closed end term that is limited to the elements recited in the claim and excludes any element, step, or ingredient not specified in the claim.

“Consisting essentially of” limits the scope of a claim to the specified materials, components, or steps (“essential elements”) that do not materially affect the essential characteristic(s) of the claimed invention. In some embodiments, the essential characteristics are the basic and novel characteristic(s) of the claimed invention.

For the sake of clarity, “characterized by” or “characterized in” (together with their related forms as described above), does not limit or change the nature of whether the list of terms following it are open or closed. For example, in a claim directed towards “a composition comprising A, B, C, and characterized in D, E, and F”, the elements D, E, and F are still open-ended terms and the claim is meant to include other elements due to the use of the word “comprising” earlier in the claim.

As used herein and in the claims, “sieving array”, “artificial sieve” refers to an artificially-made structure of a microfluidic device for target particles separation or sieving purposes. In some examples, the sieving array contains slits, wells and pillars. In some examples, the pillars arranged in an array of a plurality of rows substantially in x direction and a plurality of columns substantially in y direction. In some examples, x direction and y direction are substantially perpendicular to each other. In some examples, x direction and y direction are not substantially perpendicular to each other.

As used herein and in the claims, “in fluid communication” refers to a fluid (such as at least one liquid) flowing through from one element to another, as circumstances indicate.

As used herein and in the claims, “connect”, “connecting”, “connected” means directly or indirectly physically bound to other elements.

As used herein and in the claims, “array” is a patterned arrangement of similar objects (such as pillars), usually in rows and columns. In some examples, multiple pillars are arranged in an array form in multiple rows and multiple columns. For clarity's sake, an array can be regularly patterned (e.g., having multiple pillars with similar size and shape arranged in a regular pattern, having similar pitch, well depth and slit depth across the entire array) or irregularly patterned (e.g., having multiple pillars with similar or different sizes and shapes, arranged in an irregular pattern, having different/gradual changes in pitches, well depths and slit depths across the entire array).

As used herein and in the claims, “microfluidic device” refers to a system that manipulates small volumes of fluids (e.g., microliters to picoliters) in fabricated chambers, reservoirs, and/or microchannels, etc.

As used herein and in the claims, “sort”, “sorting”, “sorted” refers to separating, fractionating, or categorizing objects such as particles. In some examples, sorting refers to size fractionating of particles. When the target particles are sorted or separated from other non-target elements, they can be regarded as purified from the other elements.

As used herein and in the claims, “particles” are molecules or target entities which may be present in a sample and substantially suspended in a fluid. In some examples, particles are bio-particles, such as but not limited to, EVs, cells, organelles, nucleic acids, proteins and/or non-bioparticles, such as but not limited to, polymer or plastic beads, drug delivery nanoparticles, etc. In some examples, particles include but not limited to one or more of the following: liposomes, nanoparticles (e.g., polymeric, lipid-based, inorganic), protein aggregates, quantum dots, carbon nanotubes, metal particles, colloidal particles, nanowires, microplastics/nanoplastics, viruses and viral vectors, etc. In some examples, the sizes of the particles are ranged from nano-meters to micro-meters. In some examples, the bio-particles are negatively charged,

As used herein and in the claims, a “well” is a space defined between two adjacent columns of pillars, which allows fluid and particles flow therethrough.

As used herein and in the claims, “slit forming unit” is a physical microstructure which define the slit with two adjacent pillars. In some examples, the slit forming unit extended or formed from, or attached to a substrate (i.e., disposed on a substrate). In some other examples, the slit forming unit extended or formed from, or attached to a pillar (i.e., disposed on a pillar). In some other examples, the slit forming unit extended or formed from, or attached to a cover (i.e., disposed on a cover). In some other examples, the sieving array does not comprise slit forming units. The slit forming units and the pillars can be made with the same or different materials.

As used herein and in the claims, “slit” is a space defined by a slit forming unit disposed between two adjacent pillars in the same column, which allows fluid and particles flow therethrough. In some examples, the slit is in fluid communication with two adjacent wells.

As used herein and in the claims, “pillar” is a physical microstructure which define the well and the slit with the slit forming unit. In some examples, the pillar is extended or formed from, or attached to a substrate (i.e., disposed on a substrate). In some examples, the pillar substantially does not allow particles or fluid flowing therethrough. In some examples, the pillar is substantially in rectangular prism or cubic shaped. In some examples, the pillar is in other shapes such as triangular prisms, pentagonal prism, cylindrical shaped, irregular shaped etc.

As used herein and in the claims, “extracellular vesicles (EVs)” are membrane-enclosed transport carriers secreted by cells that carry a variety of biomolecules such as nucleic acids and proteins.

As used herein and in the claims, “inlet” is a port or a structure where the fluid enters the sieving array. In some examples, inlet further contains or directly or indirectly connected with or in fluid communication with one or more sample reservoirs.

As used herein and in the claims, “outlet” is a port or a structure where the fluid exits the sieving array. In some examples, outlet further contains or directly or indirectly connected with or in fluid communication with one or more collection reservoir.

It is to be understood that terms such as “top”, “bottom”, “upper”, “lower”, “left”, “right”, “middle”, “side”, “length”, “inner”, “outer”, “interior”, “exterior”, “outside”, “vertical”, “horizontal” and the like as may be used herein, merely describe points of reference and do not limit the present invention to any particular orientation or configuration. It is to be understood that directions such as “x”, “y”, “z” and the like as may be used herein, are relative directions of reference and do not limit the present invention to any particular orientation or configuration. Further, terms such as “first”, “second”, “third”, etc., merely identify one of a number of portions, components and/or points of reference as disclosed herein, and likewise do not limit the present invention to any particular configuration or orientation.

According to some of the embodiments of the subject invention, a gel-free and label-free method is provided for high-performance size fractionation of EVs-based on two-dimensional electrophoresis in an artificial sieve. The artificial sieve induces different EP mobilities for EV subpopulations based on their size, resulting in their separation as they migrate through the sieving array. The differential mobility between EV subpopulations and contaminants such as free proteins and short nucleic acids allows for the concurrent size fractionation and purification of EVs with high performance. The highly efficient and robust system of the subject invention enables the continuous-flow isolation of distinct subpopulations of EVs in a single artificial sieve device with facile sample collection, offering immense potential for EV sample preparation and point-of-care applications.

Although the description referred to particular embodiments, the disclosure should not be construed as limited to the embodiments set forth herein.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Materials and Methods

### Experimental Section

Device Fabrication. According to the embodiments of the subject invention, the artificial sieve device is fabricated on 4-inch silicon wafers using a two-mask UV lithography process. A 5 μm thick layer of low-temperature oxide (LTO) is deposited on the wafers by low-pressure chemical vapor deposition (LPCVD). Standard UV lithography and advanced oxide etching (AOE) are used to pattern trenches into the LTO layer, forming the microslits. The UV lithography and AOE steps are repeated with a second mask to create the sample reservoirs and the microwells. The LTO layer is sufficiently thick to sustain a high-voltage operation. The fabricated chip is imaged using a scanning electron microscope (JSM-6490, JEOL Ltd., Tokyo, Japan) at 10 kV. Polydimethylsiloxane (PDMS) slabs with fluidic inlet and outlet ports and electrical access vias are prepared and bonded to the artificial sieve devices after activating the surfaces in oxygen plasma (29.6 W Harrick Plasma, 65 s).

Reagents and materials. Polystyrene (PS) fluorescent particles with a diameter of 70 nm, 120 nm, 200 nm, and 300 nm are obtained from Baseline Chromtech (Tianjin, China). Ovalbumin Alexa Fluor™ 488 Conjugate, double-stranded DNA fragments 500 bp, and PKH26 membrane dye (Excitation/Emission 551/567 nm) are purchased from Thermo Fisher Scientific (Waltham, MA). Tris-borate-EDTA (TBE) buffer is obtained from Nippon Gene (Toyama, Japan). DNA intercalating dye YOYO-1 is procured from Sigma-Aldrich (Burlington, MA). Performance Optimized Polymer-6 (POP-6) is obtained from Applied Biosystems (Foster City, CA). Deionized water purified to a specific resistivity of 18.2 MΩ cm by the Direct-Q system (Millipore Corp., Bedford, MA) is used to prepare the solutions.

Preparation of test samples. Mixtures of PS particles are prepared by mixing and diluting particles of different sizes with 0.5×TBE buffer at various particle concentrations. Specifically, a binary mixture of particles of size 70 nm (2.65×10¹³ per mL) and 300 nm (3.86×10¹¹ per mL) and an additional binary mixture of size 70 nm (2.65×10¹³ per mL), and 120 nm (5.29×10¹² per mL) and a ternary mixture of particles of size 70 nm (1.76×10¹³ per mL), 200 nm (7.57×10¹¹ per mL), and 300 nm (2.24×10¹¹ per mL) are prepared. Under such concentration ratios, the particle streams exhibit similar fluorescence intensity for optimal observation and imaging. EV test samples are prepared from a mixture of small and large EVs derived from conditioned cell culture medium. Human lung cancer cells (NCI-H1975) are cultured in a humidified incubator at 37° C. and 5% CO₂. The culture medium contains Roswell Park Memorial Institute (RPMI)-1640 medium, fetal bovine serum (FBS with vesicles depleted by ultracentrifugation at 120 000×g for 90 min) and penicillin/streptomycin. The conditioned culture medium is collected at 70% confluence to a total volume of 50 mL. Low-speed centrifugation at 200×g for 5 min removed the floating cells and the remaining medium is centrifuged at 2,000×g for 15 min to deplete cell debris and large protein aggregates. The supernatant is then centrifuged at 10000×g for 20 min to pellet large EVs. Small EVs (<200 nm) including exosomes are harvested from the remaining supernatant by ultracentrifugation at 120,000×g for 90 min. The EV pellets for large and small EVs are mixed and dispersed in 0.5×PBS buffer at a total concentration of 1.70×10¹¹ per mL by vortexing thoroughly and then stained with PKH26 membrane dye. The 500 bp DNA is stained with the intercalating dye YOYO-1 at a dye-to-base pair ratio of 1:10 in TBE buffer. To prepare a mock sample for the EV purification test, stained EVs are mixed either with stained 500 bp DNA, or fluorescent-labelled OVA protein as the contaminants. The prepared samples are stored at 4° C. until processed through the artificial sieve device under test.

Experiment setup for size fractionation of EVs. The artificial sieve device is filled with TBE buffer containing 0.5% v/v POP-6 to suppress electroosmosis after bonding the PDMS slab to the silicon substrate by plasma treatment. Platinum wire electrodes (Leego Precision Alloy, Shanghai, China) are inserted in the reservoirs around the artificial sieve device. The sample solution is loaded into the input reservoir and a voltage from a Labsmith HVS448-8000D voltage supplier (Labsmith Inc. Livermore, CA) is applied generating two independent and orthogonal direct-current electric fields to drive the sample across the microslit-well sieving array for size fractionation. The particles or EVs in the sample are separated into distinct streams with different deflection angles according to their mobility difference and directed to corresponding outlets under the electric field. The separated subpopulations of particles or EVs are collected by pipetting from their respective reservoirs for downstream analysis. The experiments are monitored under an epifluorescence microscope (Eclipse, Nikon, Tokyo, Japan) equipped with a thermoelectric-cooled electron-multiplying charge-coupled device (EMCCD) camera (iXon3897, Andor, Dublin, Ireland) and a Nikon D-LEDI illuminator for the excitation and detection of fluorescence from the target substances. The camera is operated with Nikon NIS-Elements BR version 5.42 software. The fluorescence intensity profiles of the separated particle streams and the trajectories of EVs are analyzed by ImageJ software (NIH, Bethesda, MD). The resolution Rₛ between particle streams (streams 1 and 2) is calculated by Rₛ=0.5 (x₁−x₂) (σ₁+σ₂), where x and σ are respectively spatial position and standard deviation of the streams width derived from a Gaussian fit to the corresponding peak obtained along the sieve end. The baseline is chosen as the background fluorescence along the sieve end away from particle streams.

Nanoparticle tracking analysis. The size distribution of particles and EVs processed through the artificial sieve device is analyzed by a nanoparticle tracking analysis (NTA) system (Zeta View NTA, Particle Metrix GmbH, Meerbush, Germany). The particles are diluted to the optimal concentration for NTA measurements, and the system is calibrated using a 100 nm PS particle standard solution. The measurements are performed at 25° C. with the measurement settings according to the manufacturer's guide manual. The size distributions are obtained and analyzed by the Zeta View 8.02.28 software.

Transmission electron microscopy. Glow-discharge-activated grids (Pelco EasiGlow system, Ted Pella, Inc.) are used for transmission electron microscopy (TEM) sample preparation. The size fractionated EV sample solution is applied to the grids for 30 s and washed twice with distilled water. The grids are then stained with uranyl-acetate for 30 s and dried with a filter paper. A Talos120c TEM (Thermo Fischer Scientific) is used to image the EVs.

Following are examples that illustrate procedures for practicing the invention. These examples should not be construed as limiting. All percentages are by weight and all solvent mixture proportions are by volume unless otherwise noted.

### Results and Discussion

### Example 1A—Microfluidic Device Design and Operation

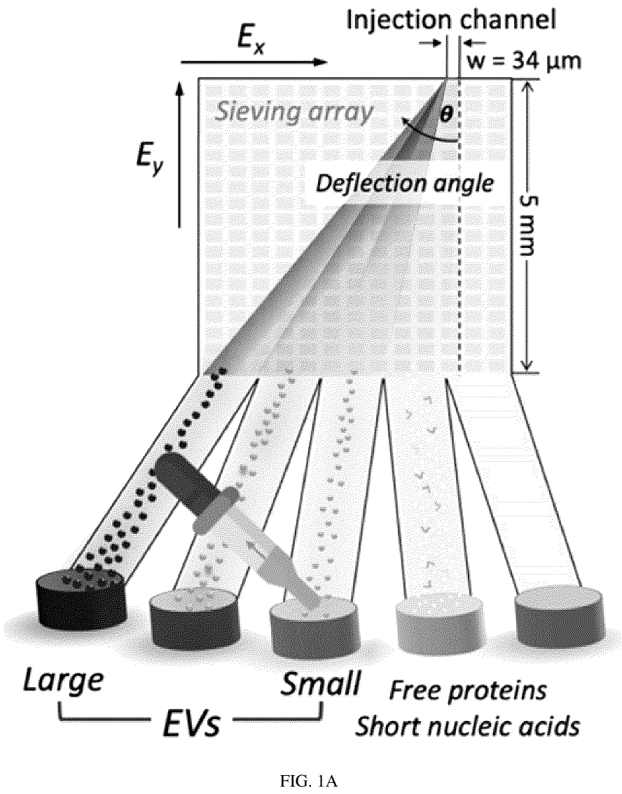

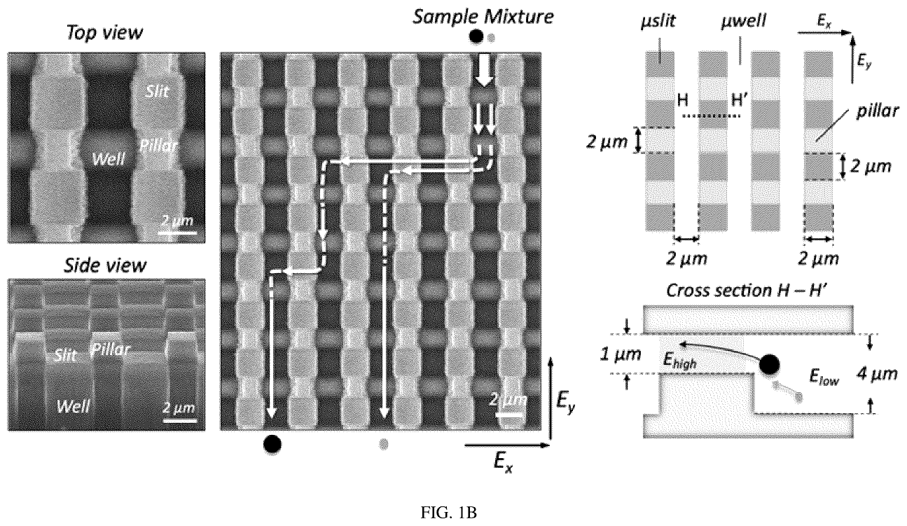

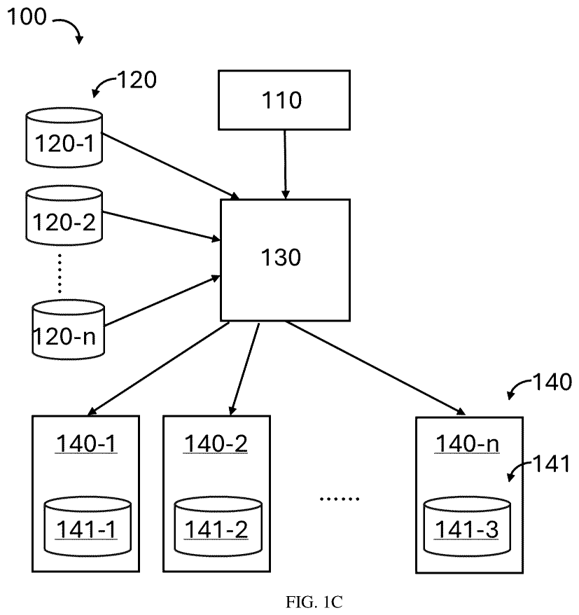

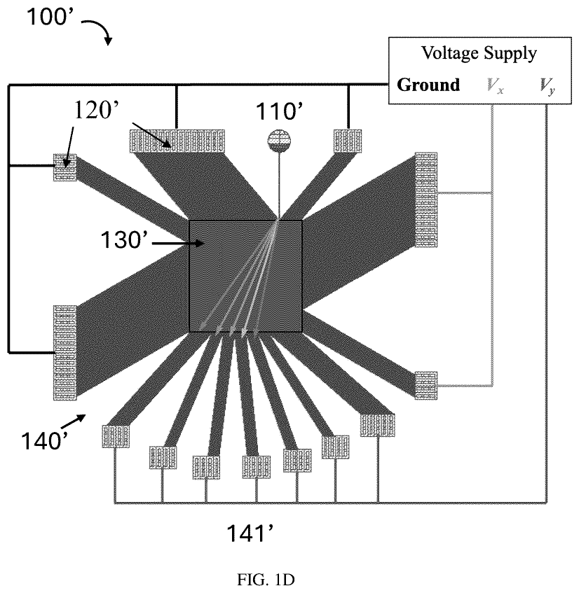

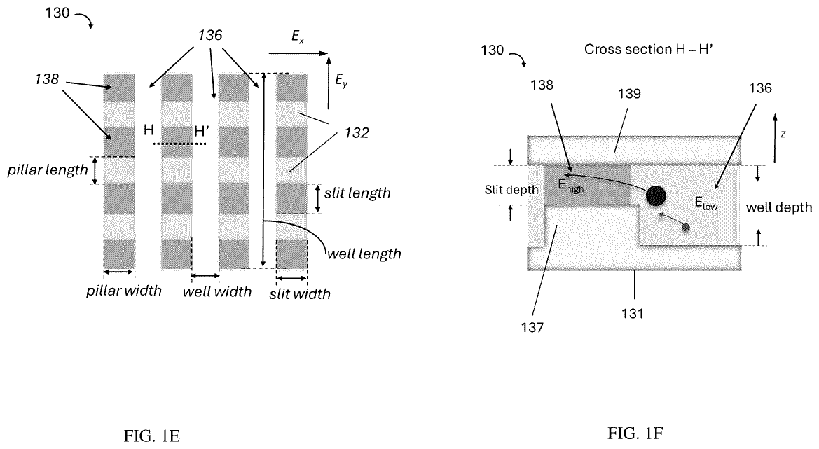

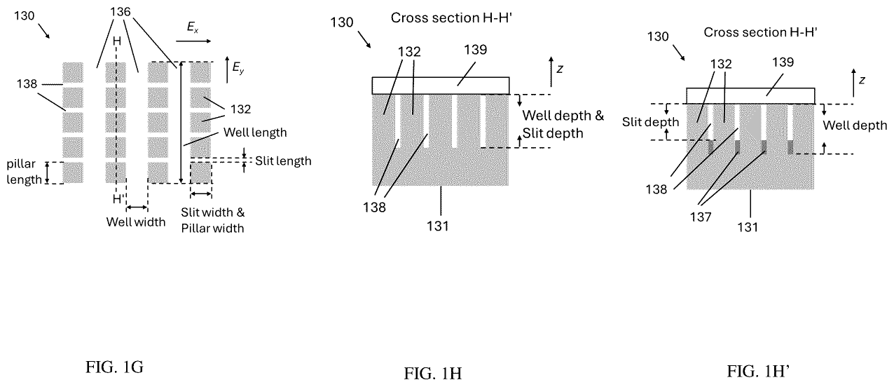

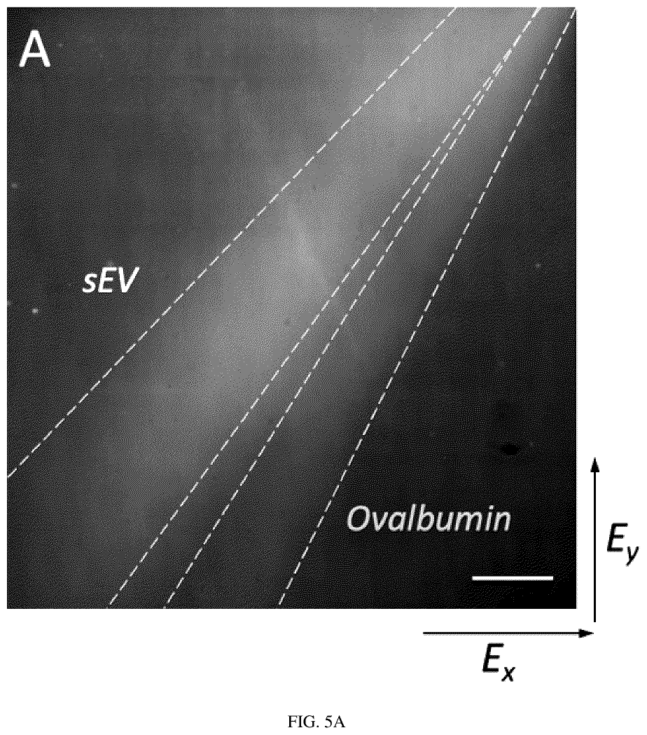

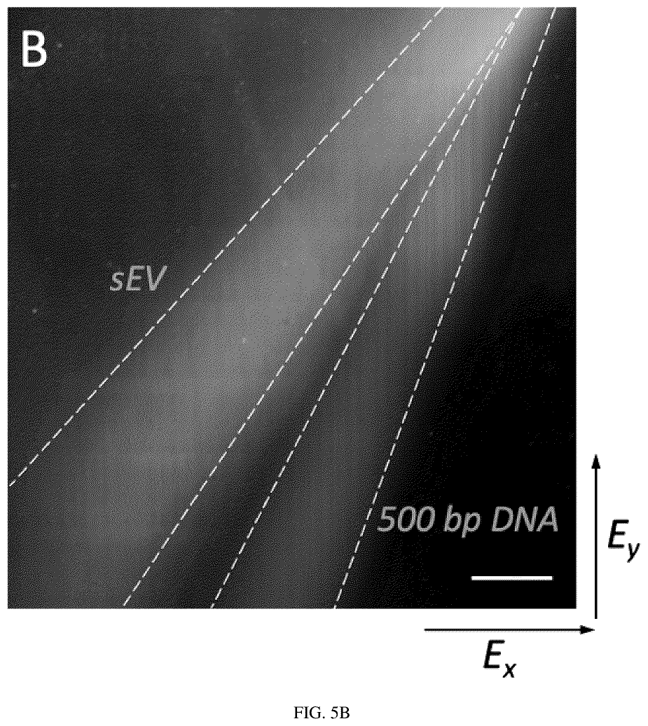

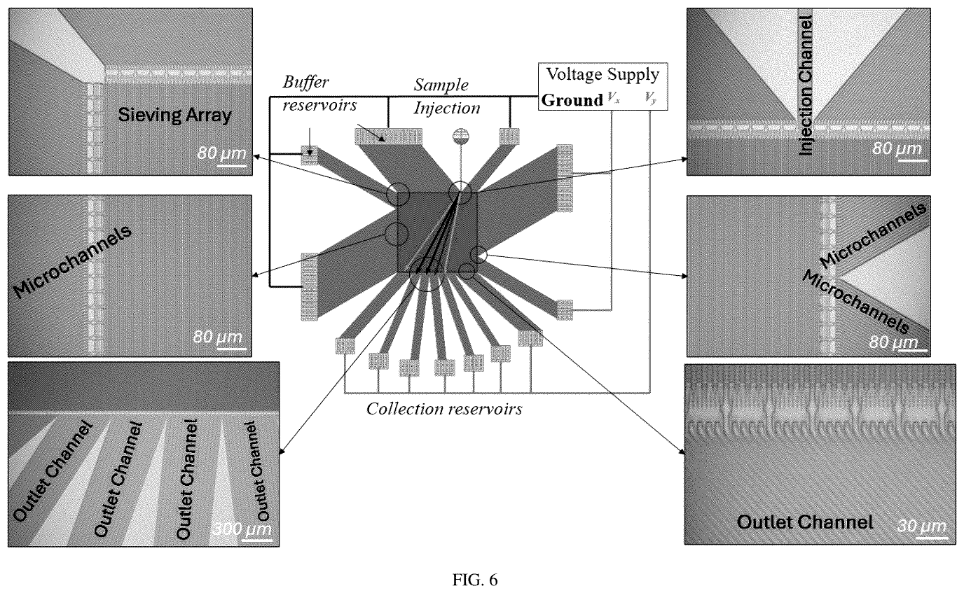

The artificial sieve device and its operation are illustrated in FIG. 1A. The microfluidic artificial sieve device comprises a 2D sieving array with a size of 5 mm by 5 mm, surrounded by microchannels for electrical and fluidic access (not illustrated), an inlet for sample loading, and multiple outlets for collecting fractionated EV subpopulations. The artificial sieve device is sealed by a polydimethylsiloxane (PDMS) slab by oxygen plasma bonding, and detailed layout and the electrical configuration are shown in FIG. 6. Electric potentials are applied through wire electrodes immersed in reservoirs and delivered to the sieving array through the surrounding microchannels to perform 2D electrophoresis. These microchannels impose a high electrical resistance and thus each serves as a current-injection source. Negatively charged components, including EVs, proteins and nucleic acids, enter the sieving array under electrophoresis. Then, the sieving array fractionates EV subpopulations based on their sizes, with larger vesicles deflecting more than smaller ones, and purifies them from soluble proteins and short nucleic acids. The fractionated EVs eventually exit through outlet ports for collection and downstream analysis.

The sieving array comprises a periodic array of microslit-wells (uSWs) with dimensions as indicated in FIG. 1B. FIG. 1B also shows the scanning electron microscopy (SEM) images of the sieving structure and schematics describing the trajectories of small and large particles across uSWs. To exert EP force on the particles, orthogonal electric fields are concurrently applied, with the E_{y} component driving the particles through the wells toward outlets and the Eₓ component directing the particles across the microslits along the x-axis direction that facilitates the size separation of the particle subpopulations. The sieving structure features over a thousand of pSWs, each functioning as a sieve. As particles migrate electrophoretically across successive uSWs, the sieving effect accumulates, ultimately resulting in separated migration paths for different subpopulations of particles. This yields particle streams that are deflected from the y-axis direction by a size-dependent deflection angle θ, with large particles experiencing more deflection than small ones. The divergence in deflection angle θ depends on the difference in the ratio of particle velocities along the x (microslits) and y (wells) directions, which originates from the particle's EP mobility across the anisotropic sieve.

### Example 1B—Example Microfluidic Device Design, Operation and Methods of Sorting Particles

### Microfluidic Device

Now referring to FIG. 1C, showing the overall structure of an example microfluidic device 100 containing an example sieving array 130 for sorting particles in a sample. The example microfluidic device 100 generally contains a sample inlet 110 for sample loading, multiple reservoirs 120, a sieving array 130, and at least one outlet 140 for collecting any sorted particles. The sample inlet 110, the at least one reservoir 120 and the at least one particle collection reservoir 140 are in fluid communication with the sieving array 130, respectively.

In this example, multiple buffer reservoirs are provided as the reservoirs (e.g., 120-1, 120-2 . . . 120-n, collectively 120) for storing buffers or other reagents, respectively. For clarity's sake, it is understood that the reservoirs 120 can be positioned in any suitable locations (e.g., top, left, right, or bottom) relative to the sieving array provided that they are in fluid communication with the sieving array.

In some examples, the sample inlet 110 is further connected and in fluid communication with an injection channel, which is positioned upstream of the sieving array 130. The sample inlet 110 is configured to import or load samples which may contain particles. In this example, the injection channel has a channel width of about 34 μm. In other examples, other suitable channel widths such as 5-100 μm or more may be used.

In some examples, multiple outlets (e.g., 140-1, 140-2 . . . 140-n, collectively 140) are connected and in fluid communication with multiple collection reservoirs (e.g., 141-1, 141-2 . . . 141-n, collectively 141) via outlet channels for collecting sorted particles, respectively. The outlets 140 and collection reservoirs 141 are positioned downstream of the sieving array 130. In some examples, two outlets are provided to collect different sizes of particles. For example, the first outlet has a channel width of about 1 mm and the second outlet has a channel width of about 4 mm. Other suitable channel widths for the outlets may be used.

Now referring to FIG. 1D, showing the overall structure of another example microfluidic device 100′ containing an example sieving array 130′ for sorting particles in a sample. The example microfluidic device 100′ generally contains a sample inlet 110′ for sample loading, multiple reservoirs 120′, a sieving array 130′, and multiple outlets 140′ connecting with multiple collection reservoirs 141′ for collecting any sorted particles. The sample inlet 110′, the at least one reservoir 120′ and the at least one particle collection reservoir 140′ are in fluid communication with the sieving array 130′, respectively. In this example, voltage supplies Vx and Vy are applied to the microfluidic device 100′ in x-direction and y-direction respectively, so that the microfluidic device 100′ can conduct 2D-electrophoreis during operation.

Now referring to FIG. 1E and FIG. 1F, showing the structure of an example sieving array. The sieving array 130 contains a substantially flat substrate 131, a top cover 139 and multiple pillars 132 which are spaced from one another and arranged in an array form, containing multiple pillar rows substantially in x direction (as indicated in arrow Eₓ) and multiple pillar columns substantially in y direction (as indicated in arrow E_{y}). In this example, the pillars 132 are disposed on the substrate 131. The pillars 132 are equidistantly spaced from each other. In other words, the pillars are arranged such that the spacing between two adjacent pillars (slit length) in the same column is the same or similar to the spacing between two adjacent pillars (well width) in the same row. Each pillar 132 has a pillar height (i.e., dimension of a pillar in z-direction), a pillar length (i.e., dimension of a pillar in y-direction) and a pillar width (i.e., dimension of a pillar in x-direction). The pillars 132 may be integral with (e.g., extended or formed from) the substrate 131 or connected with the substrate 131, and further extended to or connected with the cover 139. In this example, the pillars 132 are bonded to the cover 139. The substrate 131 and the cover 139 defines a space therebetween for the wells, the slits and the pillars. Each two adjacent columns together with the substrate 131 and the cover 139 define a well (or uwell) 136 therebetween, substantially extended along the y direction. Each column further contains multiple slit forming units 137, each of which is positioned between each two adjacent pillars 132 in the same column, such that the slit forming unit 137 and the two adjacent pillars 132 together with the substrate 131 and the cover 139 defines a slit (or uslit) 138. Each slit forming unit 137 has a slit forming unit height (i.e., dimension of a slit forming unit in z-direction), a slit forming unit length (i.e., dimension of a slit forming unit in y-direction) and a slit forming unit width (i.e., dimension of a slit forming unit in x-direction). Each well 136 has a well height (i.e., dimension of a well in z-direction), a well length (i.e., dimension of a well in y-direction) and a well width (i.e., dimension of a well in x-direction). In this example, well height is the well depth. Each slit has a slit height (i.e., dimension of slit in z-direction), a slit length (i.e., dimension of a slit in y-direction) and a slit width (i.e., dimension of a slit in x-direction). In this example, each slit forming unit 137 are extended from or attached to the substrate 131 (i.e., disposed on the substrate 131), substantially in z-direction, and the slit height is the slit depth. In other examples, the slit forming units are extended from or attached to the pillars (i.e., disposed on the pillars), substantially in x-direction or y-direction. In such case, the slit length or the slit width becomes the slit depth, respectively. In other examples, the slit forming units are extended from or attached to the cover (i.e., disposed on the cover), substantially in z-direction, and the slit height is the slit depth. For clarity's sake, the slit depth is the distance of a space between a slit forming unit and another opposing element (e.g., cover, pillar or substrate) in the direction substantially opposite to where the slit forming unit is extended or formed from, or attached to. The slit depth is configured to be smaller than the well depth. In this example, the sieving array 130 has a regular array pattern and the slit depths are substantially uniform, such as about 1 μm. Both the pillar widths and the pillar lengths arc substantially uniform, such as about 2 μm×2 μm. The pillar depths are substantially uniform, such as about the same distance of the space between the inner surface of the cover 139 and the substrate 131 or the well depth, such as 4 μm. The well depths are substantially uniform, such as about 4 μm. The well widths are substantially uniform, such as about 2 μm. The well lengths are substantially uniform and can be about the same length of the sieving array, such as about 5 mm. The well depths are substantially uniform, such as about 4 μm. Both the slit widths and the slit lengths are substantially uniform, such as about 2 μm×2 μm. The slit depths are substantially uniform, such as such as lum. The distances between two identical (e.g., central) points of two adjacent pillars (i.e., pitch) are substantially uniform, such as about 4 μm. For example, if pillar width is 2 μm, and the pillar-to-pillar distance is 2 μm, then the pitch is 4 μm.

In this example, the slits have slit length and slit width and they are substantially uniform, such as about 2 μm×2 μm, and the pitch in x-direction or y-direction is 4 μm.

In other examples, the sieving array, the substrate, or the cover have different dimensions. In other examples, the pillars, the slit forming units, slits, and/or the wells have other arrangements (either regular or irregular), other dimensions and/or spacing or pitch. For example, the sieving array, the substrate or the cover has a dimension (length×width) of about 1-10 mm×1-10 mm. For example, the well depth is about 1-8 μm, or 2-4 μm. For example, the slit depth is about 0.5-2.0 μm. For example, the pillar has a dimension (length×width×height) of about 2-10 μm×2-10 μm×2-10 μm. For example, the pitch is about 2-10 μm, or 4-20 μm. In some implementations, the slit length or slit depth is about 0.7-1.0 μm, the well depth is about 4 μm and the pitch is about 4 μm. Other suitable arrangements or dimensions may also be used. It is understood that the wells, pillars and/or slits at the peripheral sides of the sieving array may not further contain additional wells, pillars and/or slits. Instead, one or more of the wells, pillars and slits at the peripheral sides may be directly or indirectly connected with other components such as inlets, reservoirs and/or outlets, such as via channels.

Now referring to FIG. 1G and FIG. 1H, showing the structure of another example sieving array. The sieving array 130 contains a substantially flat substrate 131, a top cover 139 and multiple pillars 132 which are spaced from one another and arranged in an array form, containing multiple pillar rows substantially in x direction (as indicated in arrow Eₓ) and multiple pillar columns substantially in y direction (as indicated in arrow E_{y}). In this example, slit forming units are not provided to form the slits. Instead, the pillars are arranged such that the spacing between two adjacent pillars (slit length) in the same column is closer than the spacing between two adjacent pillars (well width) in the same row. In this example, the pillars 132 are disposed on the substrate 131. Similar to the example sieving array as shown in FIG. 1E and FIG. 1F, each pillar 132 has a pillar height (i.e., dimension of a pillar in z-direction), a pillar length (i.e., dimension of a pillar in y-direction) and a pillar width (i.e., dimension of a pillar in x-direction). The pillars 132 may be integral with (e.g., extended or formed from) the substrate 131 or connected with the substrate 131, and further extended to or connected with the cover 139. In this example, the pillars 132 are bonded to the cover 139. The substrate 131 and the cover 139 defines a space therebetween for the wells, the slits and the pillars. Each two adjacent columns together with the substrate 131 and the cover 139 define a well (or uwell) 136 therebetween, substantially extended along the y direction. In this example, each two adjacent pillars 132 in the same column together with the substrate 131 and the cover 139 defines a slit (or uslit) 138. Each well 136 has a well height (i.e., dimension of a well in z-direction), a well length (i.e., dimension of a well in y-direction) and a well width (i.e., dimension of a well in x-direction). In this example, well height is the well depth. Each slit has a slit height (i.e., dimension of slit in z-direction), a slit length (i.e., dimension of a slit in y-direction) and a slit width (i.e., dimension of a slit in x-direction). In this example, the slit height is the slit depth. The slit length is configured to be smaller than the well width. The slit depth is the same or similar to the well depth. In this example, the sieving array 130 has a regular array pattern and the slit lengths are substantially uniform, such as about lum. Both the pillar widths and the pillar lengths are substantially uniform, such as about 2 μm×2 μm. Both the slit depths and well depths are substantially uniform, such as about the same distance of the space between the inner surface of the cover 139 and the substrate 131, such as 4 μm. The well widths are substantially uniform, such as about 2 μm. The well lengths are substantially uniform and can be about the same length of the sieving array, such as about 5 mm. The distances between two identical (e.g., central) points of two adjacent pillars (i.e., pitch) in x-direction are substantially uniform, such as about 4 μm; and that in y-direction are substantially uniform, such as about 3 μm.

Now referring to FIG. 1G and FIG. 1H′, showing the structure of another example sieving array. In this example, the overall structure of sieving array 130 is similar to that as shown in FIG. 1G and FIG. 1H, but multiple slit forming units 137 are additionally provided on the substrate 131, defining the slits 138 with the pillars 132 and cover 139. In this example, the slit length is configured to be smaller than the well width, and the slit depth is configured to be smaller than the well depth. For example, the slit depths are substantially uniform, such as 3 μm. For example, the well depths are substantially uniform, such as 4 μm.

In these examples, the substrate 131 is formed from silicon wafers with a layer of low-temperature oxide (LTO) deposited onto it. The layer is sufficiently thick to sustain a high-voltage operation. In this example, the layer is about 5 μm thick. In other examples, the layer is about 1-100 μm. The slits 138 are formed by patterning trenches into the low-temperature oxide (LTO) layer by standard UV lithography and advanced oxide etching (AOE). The reservoirs and the microwells are formed by repeating the UV lithography and AOE steps with a second mask. In this example, polydimethylsiloxane (PDMS) slab is provided as the cover 139 and bonded to the device to form the sieving array 130 after activating the surfaces in oxygen plasma (29.6 W Harrick Plasma, 65 s), and fluidic inlet and outlet ports and electrical access vias are also provided and connected with the sieving array to form the example microfluidic device.

The microfluidic device is configured to fractionate the particles in the sample. In some implementations, the microfluidic device is used for size fractionation, separation or purification of extracellular vesicles (EVs) in a sample. In some implementations, EVs are used as example particles for sorting. In some examples, the sample may or may not contain one or more of the following: extracellular vesicles (EVs), proteins, nucleic acids and combination thereof. In some examples, the particles have particle sizes ranged from about 30 nm to lum, 50 nm to lum, or about 70 nm to 300 nm. In some examples, EVs in a sample include exosomes with a size range about 30-150 nm, microvesicles (MVs) with a size range about 100-1,000 nm and/or apoptotic bodies with a size range about 500-5,000 nm (1-5 μm), or >800 nm.

### Operation of Microfluidic Device

In this example, prior to operation, the microfluidic device is pre-filled with a buffer after bonding the PDMS slab to the silicon substrate by plasma treatment. In this example, the buffer is 0.5×tris-borate-EDTA (TBE) buffer. In some examples, the sieving array is configured to be pre-treated with an agent that is able to suppress or stabilize electroosmotic flow (EOF). In this example, the agent is included in the buffer and contains about 0.5% v/v Performance Optimized Polymer-6 (POP-6). In other examples, the agent is POP-6, POP-4, POP-7 or polyethylene oxide (PEO), or combination thereof. In this example, platinum wire electrodes (Leego Precision Alloy, Shanghai, China) are inserted in the reservoirs around the sieving array. The sample solution is loaded into the input reservoir connected with the sample inlet and a voltage from a Labsmith HVS448-8000D voltage supplier (Labsmith Inc. Livermore, CA) is applied generating two independent and orthogonal direct-current electric fields to drive the sample across the sieving array.

In this example, during operation, particles are driven by the voltage applied to the device and pass through or migrate across one or more of the multiple slits and/or one or more of the multiple wells based on at least particle size, reaching the multiple outlets in different positions, thereby sorted particles are collected from the at least one outlet. The microfluidic device is configured to receive a first electric field substantially in x direction and a second electric field substantially in y direction, respectively. In some examples, the first electric field is about 5-1,000 Vcm⁻¹, and the second electric field is about 5-1,000 Vcm⁻¹. In some examples, the first electric field is about 40-250 Vcm⁻¹, and the second electric field is about 60-300 Vcm⁻¹. In other samples, instead of applying voltage or electric fields to the microfluidic device, other means may be used to drive the particles to sort or separate with the microfluidic device. For example, alternatively or additionally, pressure driven flow can be applied to drive particles to sort or separate.

### Methods for Sorting Particles in a Sample

In this example, provided is a method for sorting particles in a sample, comprising the steps of:
(a) providing any one of the microfluidic device as described herein;
(b) loading a sample to the sample inlet of the microfluidic device;
(c) flowing the sample into the device, such that particles are sorted and collected in the at least one outlet.

In this example, the step (c) further contains the step of: applying a first electric field in x direction and a second electric field in y direction to perform 2D electrophoresis, such that particles are sorted and collected in the at least one outlet. Prior to the step (b), further containing the step of: preparing the device by pre-filling a buffer to the sieving array. In one implementation, the buffer is 0.5X tris-borate-EDTA (TBE) buffer. In other examples, other suitable buffers with suitable concentrations can be used. In one implementation, prior to the step (b), further comprises the step of: pre-treating the device by coating surfaces of the device with an agent that is able to suppress or stabilize electroosmotic flow (EOF). In one implementation, the buffer further contains the agent as one step to preparing the device while suppressing or stabilizing EOF. For example, the agent is or contains about 0.5% v/v Performance Optimized Polymer-6 (POP-6). In other examples, other agents such as POP-4, POP-7, or PEO may be used. In one implementation, sample containing EVs subpopulations are used for size fractionation, separation or purification.

### Example 2—Validation with Polystyrene Particles

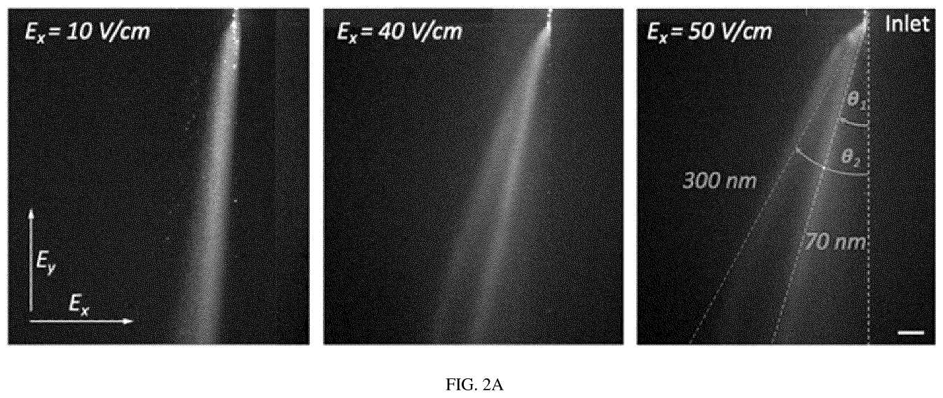

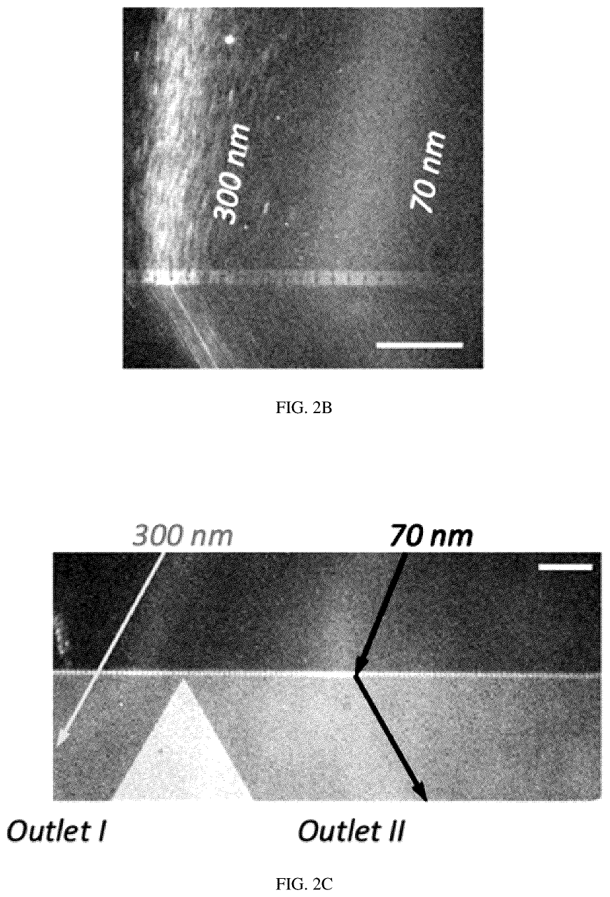

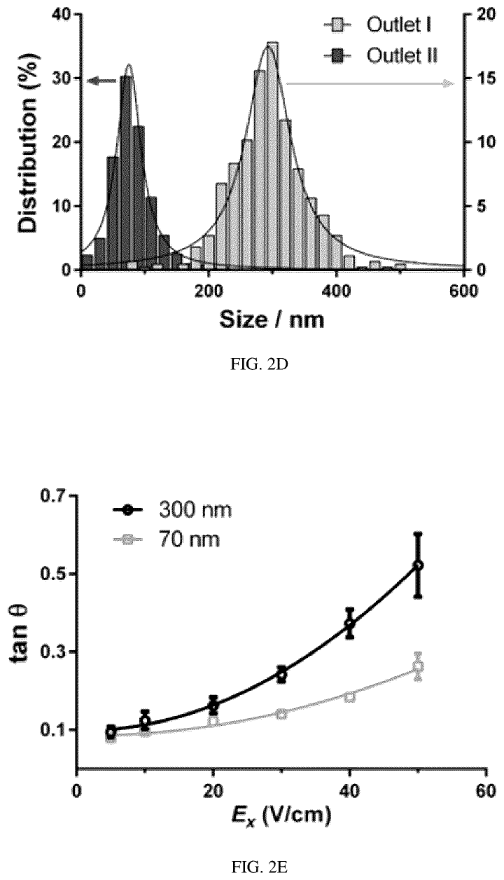

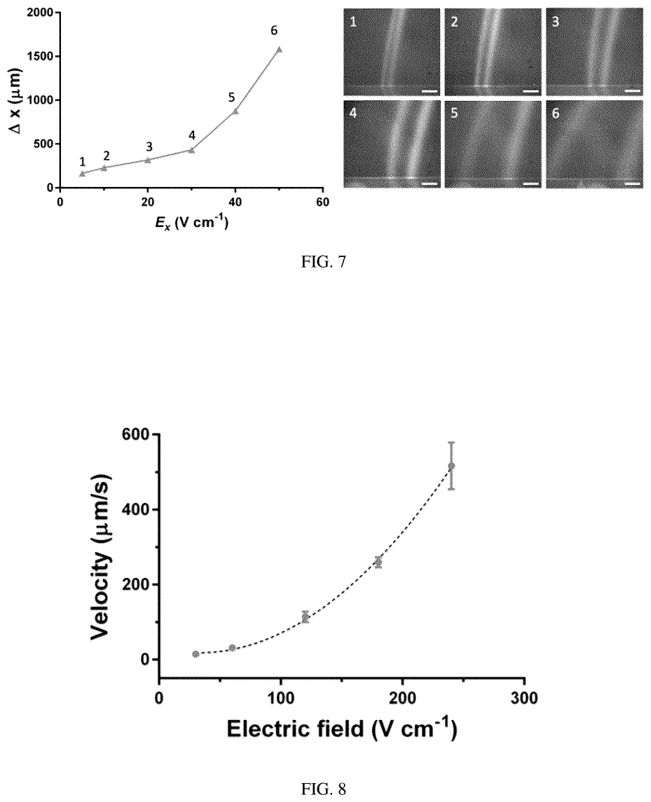

The design of the artificial sieve device is validated by a binary suspension of polystyrene (PS) particles with diameters of 70 nm and 300 nm. The particles with a diameter of 70 nm have a similar size to the exosomes (one of the EV subpopulations), and the particles with a diameter of 300 nm represent EVs with larger sizes. The experimental results are obtained by the artificial sieve device of the subject invention (1 μm deep slits and 4 μm deep wells, pitch 4 μm) filled with TBE buffer. The particle suspension is first introduced into the sample reservoir and then injected into the sieving array by applying two orthogonal electric fields/y and E_{y}. FIG. 2A shows the separation of 300 nm and 70 nm fluorescent particles under varying/while keeping by constant at 60 V cm⁻¹. When Eₓ=20 V cm⁻¹, the separation already occurs which is confirmed by examining the particle streams at the sieve end using a high magnification lens as shown in FIG. 2B. The 300 nm particles can be individually spotted while the 70 nm particles are not discernable due to their nanometer scale small size. As the Eₓ increases further, the separation of the particles into two distinct streams becomes more discernible with an increased deflection angle difference between the two streams. Under Eₓ=50 V cm⁻¹, E_{y}=60 V cm⁻¹, the 70 nm particle stream follows a trajectory with a small deflection angle θ₁ at 16.5°, while 300 nm particles migrate further along the x-axis direction with a larger deflection angle θ₂ at 31.1°. The rapid expansion of mixture stream at the injection point could be an indication of a distorted electric field at the interface of the injection microchannel and the sieving array. Such distortion could be minimized by optimizing the design of sample injection microchannel and surrounding microchannels. Nevertheless, the streams do not show significant dispersion or broadening in the downstream direction, indicating that the particles remain in their compact stream during the separation. The particle streams exit through the collection ports at the end of the sieving array as shown in FIG. 2C. The increase of Eₓ also enhances the separation distance (Δx) of the two particle streams at the sieve end from <200 μm at Eₓ=5 V cm⁻¹ to >1.5 mm at Eₓ=50 V cm⁻¹ as shown in FIG. 7. The large separation distance allows the collection of the separated particles from designated outlets with minimal cross contamination. The trajectories of particle streams near the sieve end as shown in FIG. 7 demonstrate slight curvatures which could be addressed by optimizing the design of the surrounding current-injection microchannels to attain a more uniform field. To examine the particles fractionated through the device, size distribution of the collected particle subpopulations from outlets is measured by NTA. The size distribution exhibits two peaks at 70 nm and 300 nm for those particles collected from outlet I and II respectively, which coincides with the input particle sizes as shown in FIG. 2D.

The stream deflection angle θ reflects the size-dependent mobility of the particles in the x direction. The slope of the stream as tan θ, under increasing Eₓ for both 70 nm and 300 nm particles while keeping E_{y} constant at 60 V cm⁻¹ is plotted in FIG. 2E. When Eₓ is increased from 5 V cm 1 to 50 V cm⁻¹, the deflection angle θ (tan θ) increases for both streams. It is worth mentioning that both particle sizes show a non-linear trend of tan θ with respect to Eₓ. The tan θ can be expressed as the ratio of the particle's EP velocity (v=μE) along the x- and y-axis directions:

$$tan θ = {μ}_{x} {E}_{x} / {μ}_{y} {E}_{y} \qquad ( 1 )$$

where μₓ and μ_{y} are the particle's respective EP mobilities. This non-linear behavior of tan θ can be attributed to the particles having a field-dependent mobility. The mobility is typically considered a constant, but it can also depend on the field strength for DNA, cells, and particles.³⁹⁻⁴² The field-dependent mobility of 300 nm particles is validated by analyzing the particle's motion under varying E_{y} in the absence of Eₓ as shown in FIG. 8. If k is defined as the factor of the field dependence, then the mobility along x-axis direction can be expressed as μₓ=μ₀+kEₓ, where μ₀ is the mobility at E≈0 and Eₓ is the magnitude of the field. If Eₓ is increased while keeping E_{y} constant, then:

$$tan θ = ( {μ}_{0} + k {E}_{x} ) {E}_{x} / {μ}_{y} {E}_{y} \qquad ( 2 )$$

In this regime, tan θ increases quadratically with respect to Eₓ. The fitting curve is plotted and the relation is found to match the experimental data with R²>0.99 for 300 nm particles and 70 nm particles. The mobility of 70 nm particles across the uSWs along the x-axis direction also exhibits a non-linear relationship with the applied voltage. However, the increase in mobility is less pronounced at higher voltages, suggesting a smaller k factor for 70 nm particles.

Unlike the conventional EP platforms with uniform electric field distributions, the pSW structure of the subject invention creates an anisotropic electric field pattern, which is crucial to induce differential EP velocity for particles migrating along x and y axes. The particles migrate along the y-axis direction through the microwells, in which the field has a relatively uniform and low strength. This contributes to a comparable velocity along the y-axis of both 70 nm and 300 nm particles. Along the x-axis direction, however, the uSW structure exhibits high field strength regions when crossing the microslits. Benefiting from the nonlinear electrophoresis in strong electric fields, there is an increase of EP velocity vx near the microslits that is more dominant for larger particles than smaller particles. The high field region near a microslit acts as a velocity accelerator to push large particles into the microslit more readily than small particles. This results in large particles crossing more microslits than small particles as they travel through the microwells. In other words, this drives large particles towards the direction of larger deflection angle θ, thereby separating them from smaller ones.

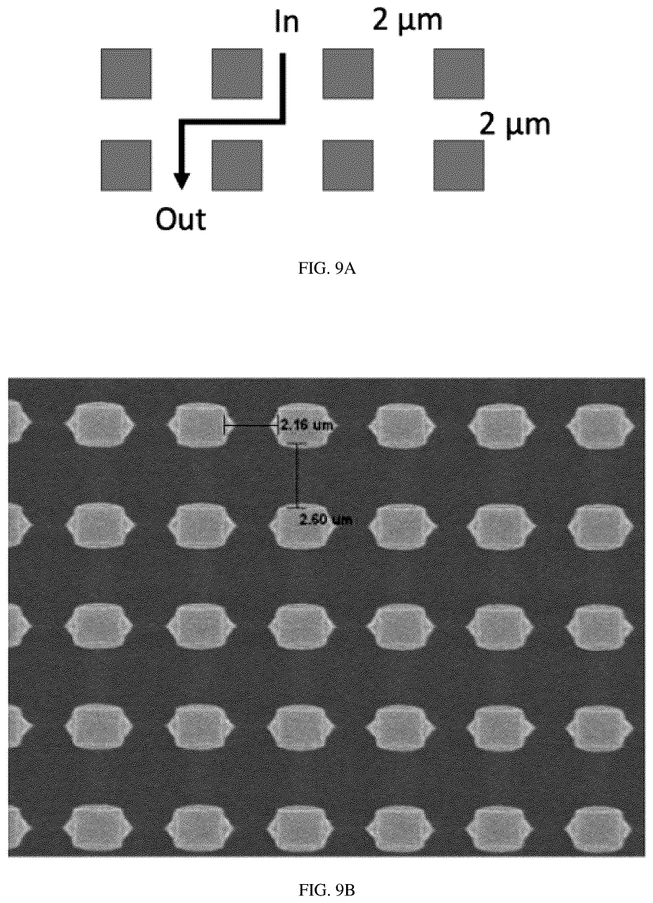

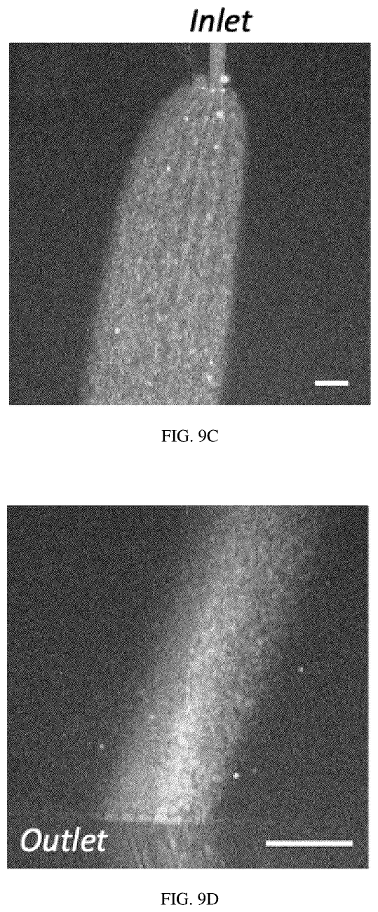

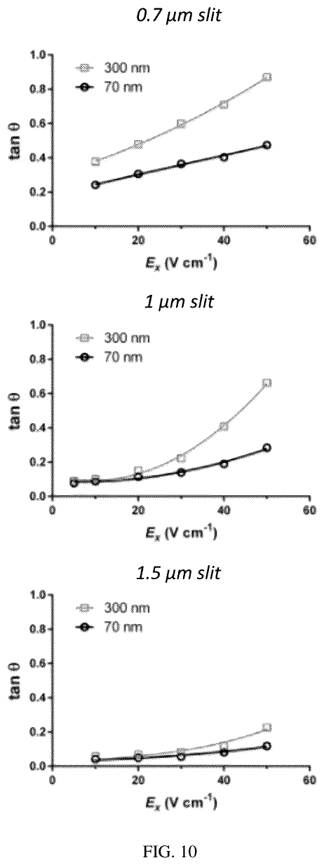

As a negative control, an artificial sieve device of the subject invention with a micropillar array that generates an isotropic electric field pattern along the x and y axes is also fabricated to further validate the pivotal effect of non-uniform electric field on the particle separation as shown in FIG. 9. As expected, this artificial sieve device did not exhibit any noticeable separation of the particles. To further investigate the effect of the slit depth on the particle separation, devices with different slit depths of 0.7, 1 μm and 1.5 μm are fabricated, while keeping the well depth constant at 4 μm. Increasing electric fields Eₓ are applied while keeping E_{y} constant, and a non-linear increase of the tan θ with Eₓ is also observed in devices with 0.7 and 1.5 μm slits as shown in FIG. 10. At Eₓ 50 V cm⁻¹, and E_{y} 60 V cm⁻¹, the device with 1.5 μm slit depth resolves 70/300 nm particle mixture into two distinct streams with a deflection angle difference of Δθ=6.0°, whereas for 1 μm and 0.7 μm slit devices, the Δθ are 14.6° and 15.7° respectively. The results show that narrowing the slit depth improves the separation resolution. However, further reducing the slit depth could cause collapse (during PDMS bonding) and particle clogging, which will affect the overall fractionation efficiency. Therefore, the device with 1 μm deep slits and 4 μm deep wells is used for the experiments.

In addition to the device design, the working parameters are also optimized to enhance the EP performance. To suppress the electroosmotic flow (EOF), the artificial sieve device surface is coated with the performance optimized polymer POP-6. This dynamic coating effectively suppresses EOF to ensure desired EP performance. An optimal concentration of 0.5% v/v POP-6 is used, as higher concentrations interfere with the surface charge of the particles, and lower concentrations are unable to suppress EOF. EP performance is also affected by the ionic strength of the buffer. As shown in FIG. 11, the EP velocity of 70 nm particle increases with the dilution of the buffer. With increased ionic strength, EP velocity is reduced and limits the overall throughput. Moreover, particles in overdiluted TBE buffers (for example, 0.05×TBE) are unable to inject under electric field (not shown) because the EOF is dominating in the lower ionic strength buffer. Therefore, the working buffer 0.5×TBE is chosen for the best EP performance.

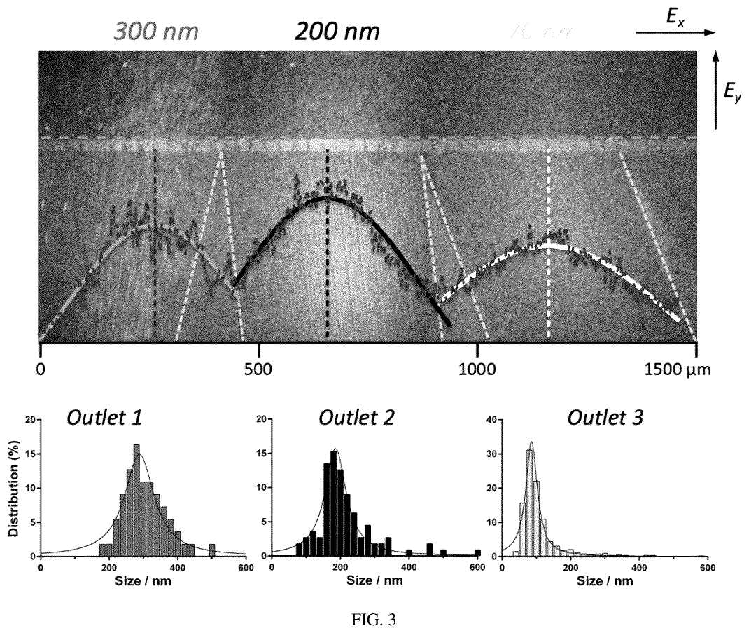

After optimizing the device design and the working parameters, the size fractionation of a ternary mixture of particles having diameters of 70 nm, 200 nm and 300 nm is investigated. The artificial sieve device can resolve the mixture into three distinct particle streams and directed individual streams to the corresponding outlets for collection, as shown by the fluorescent image in FIG. 3. The resolution of the size fractionation is evaluated by measuring the separation distance and the resolution factor (Rₛ) between different particle streams. The separation distances are 410 μm between the particle streams of 300 nm and 200 nm, and 525 μm between 200 nm and 70 nm streams. This corresponds to the Rₛ values of 0.48 and 0.51. The NTA analysis of the size distribution of the collected fraction verifies the size fractionation of the ternary mixture. The sieving method of the subject invention offers a one-pot particle size fractionation at sub-micrometer resolution, which outperforms other platforms such as micro-filtration that requires replacement of filtration membranes for each target particle size.

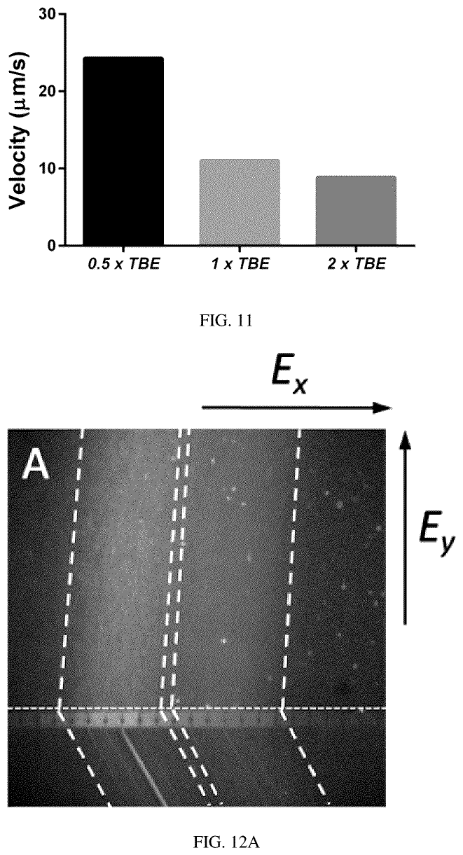

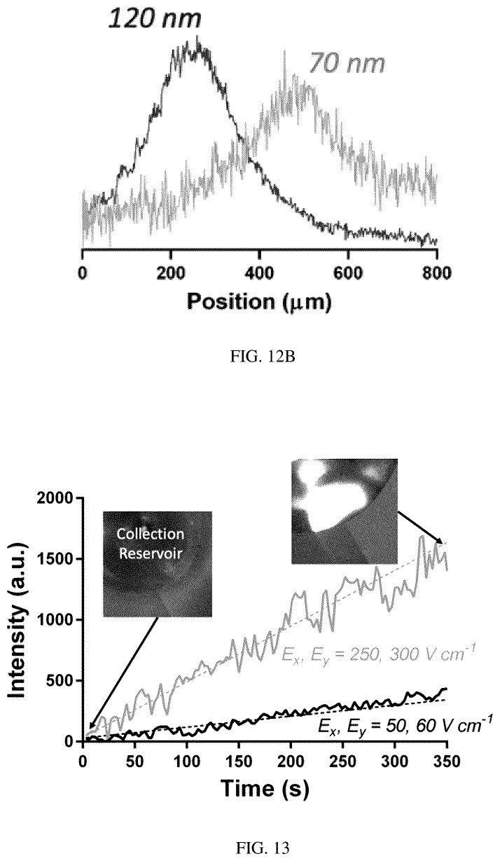

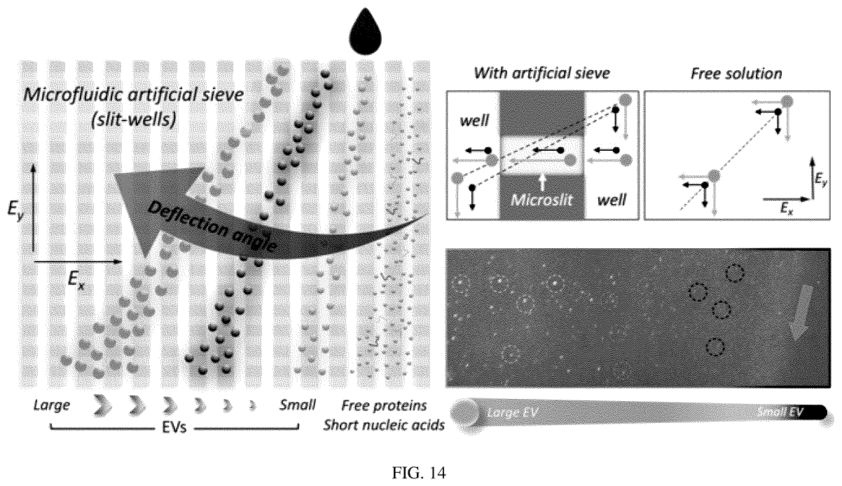

The separation resolution is an important factor that determines the sieving performance for size fractionation of certain subpopulations within a small size range from a particle mixture. The separation resolution of the uSW-based chip is evaluated using a mixture of 70 nm and 120 nm particles. Despite the small size difference, the particle mixture can be successfully resolved into distinct streams as shown in FIGS. 12(A)-12(B). The plot of fluorescence intensity near the outlet channels along the x-axis direction shows lateral isolation of the two streams with a minimum resolvable size of 50 nm and a resolution value Rₛ of 0.35. The peaks of the two streams have a wide gap of ˜ 300 μm; however, a minor cross contamination is still present. This is mainly due to the particle dispersion in the stream and the significant diffusion of small particles. The amount of spreading in the stream of 70 nm particles in FIGS. 12(A)-12(B) and FIG. 3 shows noticeable difference. This is likely contributed by the interference from 200 and 300 nm particles in the ternary mixture as opposed to the interference from 120 nm particles alone in the binary mixture. A potential method to enhance the device resolution is to reduce the pitch size or introduce more μSW structures to the sieving array.

### Example 3—High Performance Size Fractionation of EVs

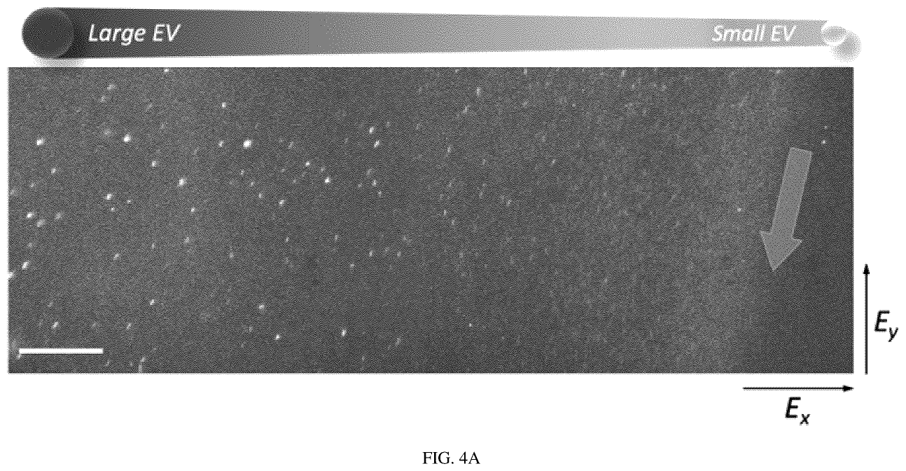

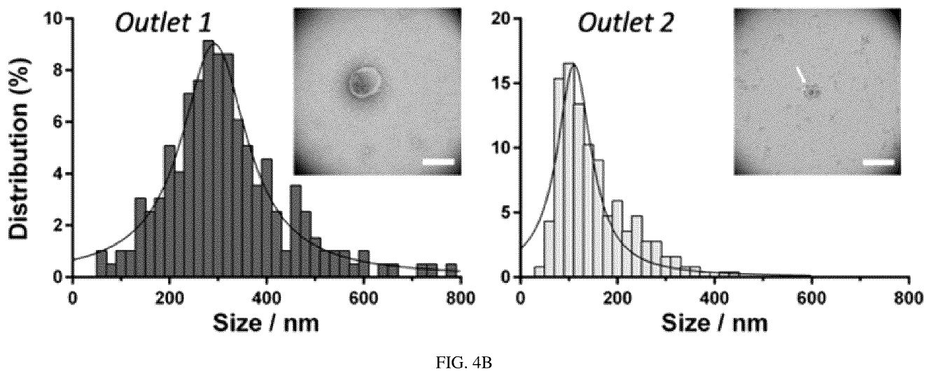

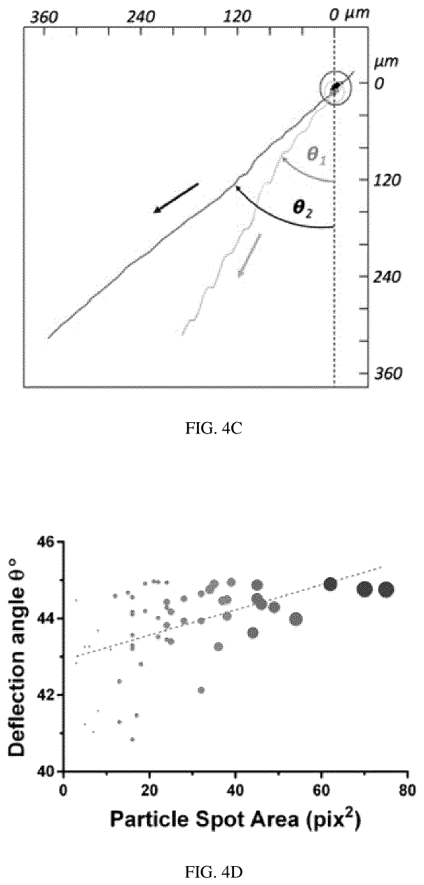

The size fractionation performance for cell-derived EVs is evaluated using EVs obtained from human lung cancer cells (NCI-H1975). EVs are prepared by high-speed centrifugation and ultracentrifugation to obtain large-size and small-size EVs, respectively. After mixing the EVs thoroughly and staining them with PKH membrane dye, their trajectories across the μSWs are observed on-chip and recorded for further analysis. FIG. 4A shows the fractionation of EVs across the sieving array. Unlike PS particles, which show a bright and focused stream owing to their relatively uniform size, the EVs are highly heterogeneous in size and distributed sparsely across the sieving array during fractionation. Nevertheless, the size distribution exhibits a clear pattern that deflection angle θ increases with EV size, and large EVs migrate further in x direction. The fractionated EVs from the artificial sieve device of the subject invention could originate from apoptotic bodies (>800 nm), microvesicles (100-1000 nm) or exosomes (30-150 nm, rightmost stream, least deflection). These results demonstrate that the artificial sieve device of the subject invention can fractionate EVs with a wide size range. The size distribution of the collected EVs is analyzed by NTA and histograms are shown in FIG. 4B. The small EVs or exosomes that have a small deflection angle (the brighter stream on the right in FIG. 4A) are collected from outlet II at the right position, and the size distribution of the collected particles shows a peak at 100 nm, which corresponds to small EVs and exosomes. The distribution is slightly broad with some EVs having larger sizes, which could be attributed to the fact that the sieving is based on the EP mobility which may not only depend on the size. The mobility variation due to the heterogeneous properties of EVs, such as surface charge and contents will have an influence on the uniformity of size fractionation. Larger vesicles having a large deflection angle are directed to outlet I, which exhibits a wide size distribution with a peak around 300 nm. Negative-staining electron microscopic images of EV subpopulations show intact morphology in round shape, consistent with previous reports using other methods as shown in FIG. 4B.

Fractions of small and large EVs collected from the sieve show relatively broad size distributions as shown in FIG. 4B in comparison to those of PS particles separated through the sieve as shown in FIG. 3. For instance, purity (defined as the percent fraction of recovered particles that fall below 200 nm size) is 93% in the case of 70 nm particles whereas this value is 80% in the case of small EVs. This observation could be attributed to several factors. One factor could be dispersion of streams that can lead to crosstalk among adjacent outlet channels. Adjusting outlet dimensions or further splitting outlet channels may help increase purity. Purity can also be increased by reducing stream dispersion through an optimized sieve design and the separation resolution at the sieve end could be enhanced by increasing the overall sieve size. Another potential factor that leads to broad size distribution of collected small EVs is their heterogeneity. Unlike synthesized particles as shown in FIGS. 2 and 3 with relatively uniform physical properties, EVs may differ in their shape, surface charge, origin and content which may have a direct influence on their EP mobility. A further factor to consider is the possible presence of protein and EV aggregates, and small EV debris. Similar broad peaks are also noted for EVs separated through capillary electrophoresis. FIG. 4C shows the EP trajectories of two individual EVs across μSWs. An artificial sieve device with 10 μm pitch is employed for better visualization of their movement. The zigzag profile of both EV trajectories is attributed to the periodical pattern of μSWs. The size-dependent separation of EVs by 2D electrophoresis is further illustrated by the plot of the optical cross-sectional area (indicative of relative size) versus the deflection angle of single EVs, as shown in FIG. 4D. The overall deflection angle θ increases as the particle size increases; these results are consistent with the PS particle experiments. The fractionated EVs are directed to the collection outlets under electrophoresis and the fluorescence intensity of the outlet reservoir over time is measured indicating continuous accumulation of target EVs as shown in FIG. 13. The sieving mechanism also remains functional at an increased electric field strength of 250 V cm⁻¹, which facilitates higher fractionation efficiency and demonstrates the device's ability to operate in a wide voltage range. Operating at a high electric field strength and voltage is desired because doing so accelerates the streams and thus reduces their dispersion (less time for diffusion), thereby increasing the separation efficiency and resolution. This is required for resolving the streams of particles with minimal size difference as in the case of EVs.

### Example 4—EV Purification from Protein and Short Nucleic Acid

The purification of EVs from other abundant components such as soluble proteins and cell-free nucleic acids (NAs) in blood or serum is challenging due to their nanoscale dimensions. Conventionally, this can be realized using ultracentrifugation, but the process is tedious and time-consuming. The capability of the artificial sieve of the subject invention to purify EVs from proteins and Nas is evaluated. Similar to the EVs which possess negative surface charge due to their phospholipid membrane, proteins and NAs also exhibit negative charge based on their specific chemical groups. Therefore, they can be co-injected into the device along with EVs under electrophoresis. Nonetheless, the size and charge of proteins and NAs differ from those of EVs, which can lead to varying mobilities across the μSW sieving array and distinct deflection angles, thus enabling the purification of EVs. The device's capability of purifying EVs from soluble proteins or NAs is investigated by using a sample mixture of small EVs (<200 nm) and either ovalbumin (OVA, 45 kDa) as a model soluble protein or 500 bp DNA as a model cell-free NA. The small EVs stream exhibits a larger defection angle and is separated from OVA stream under Eₓ 60 V cm⁻¹, E_{y}=60 V cm⁻¹, as shown in FIG. 5A. The artificial sieve device can also fractionate small EVs from 500 bp DNA stream, demonstrating its potential to isolate EVs from short nucleic acids, such as cell-free DNA (cfDNA), which typically ranges from 50 bp to 300 bp in length as shown in FIG. 5B. Under the same optimized operating parameters, the deflection angles for different target substances in a biofluid are in following orders: θ_{largeEV}>θ_{smallEV}>θₚᵣₒₜₑᵢₙ˜θ_{cfDNA}. This information can help optimize the outlet channel design to collect specific subpopulations from a complex biofluid with precision. Further optimization of the sieve array may involve tuning the dimensions of slits, wells, and surrounding microchannels for a more uniform electric field distribution across the sieve and reduced sample dispersion. It could also involve reducing the array pitch and increasing the array size for an enhanced separation resolution. In the current architecture, slits are aligned in rows to keep a relatively simple layout design. Other arrangements and architectures presenting an anisotropic electric field can be explored for the size-fractionation of particles of EVs.

The effective method for size fractionation of EV subpopulations using 2D electrophoresis in a microfluidic artificial sieve is provided. The artificial sieve device exploits the size-dependent mobility of EVs under an anisotropic electric field pattern to achieve high-resolution fractionation and concurrent removal of nanoscale contaminants. The artificial sieve device also enables continuous flow operation and facile sample collection, which are desirable features for practical applications. The artificial sieve device can be readily integrated with other microfluidic modules or analytical instruments for downstream processing and characterization of EV subpopulations. The platform of the subject invention also provides a significant advancement in the field of EV research and opens up new possibilities for discovery of new EV types, identifying new biomarkers for specific EV subtypes and diagnosis of various diseases based on EV subpopulations.

Extracellular vesicles (EVs) are cell-derived particles that exhibit diverse sizes, molecular contents and clinical implications for various diseases depending on their specific subpopulations. However, fractionation of EV subpopulations with high resolution, efficiency, purity, and yield remains an elusive goal due to their diminutive sizes. In this study, we introduce a novel strategy that effectively separates EV subpopulations in a gel-free and label-free manner, using two-dimensional (2D) electrophoresis in a microfluidic artificial sieve. The microfabricated artificial sieve comprises periodically arranged microslit-well structures in a 2D array and generates an anisotropic electric field pattern to size fractionate EVs into discrete streams and steer the subpopulations into designated outlets for collection within a minute. Along with fractionating EV subpopulations, contaminants such as free proteins and short nucleic acids can be simultaneously directed to waste outlets, thus accomplishing both size fractionation and purification of EVs with high performance. The platform of the subject invention offers a simple, rapid, and versatile solution for EV subpopulation isolation, which can potentially facilitate the discovery of biomarkers for specific EV subtypes and the development of EV-based therapeutics.

### Numbered Embodiments

Embodiment 1. A microfluidic device for sorting particles in a sample, comprising: a sample inlet for sample loading; at least one reservoir; a sieving array; and at least one outlet for collecting any sorted particles, wherein the sample inlet, the at least one reservoir and the at least one particle collection reservoir are in fluid communication with the sieving array, respectively, wherein the sieving array comprises a substrate and a plurality of pillars, the plurality of pillars are spaced from one another and arranged in an array of a plurality of rows substantially in x direction and a plurality of columns substantially in y direction, wherein each two adjacent columns define a well therebetween, thereby forming a plurality of wells in the sieving array, wherein each two adjacent pillars in the same column further define a slit therebetween, thereby forming a plurality of slits in the sieving array, such that, when in operation, particles are driven to pass through one or more of the plurality of slits and/or one or more of the plurality of wells based on at least particle size, thereby particles are sorted and collected from the at least one outlet.

Embodiment 2. The device of embodiment 1, wherein each well has a well depth and a well width, and each slit has a slit depth and a slit length; and wherein the slit depth is configured to be smaller than the well depth; and/or the slit length is configured to be smaller than the well width.

Embodiment 3. The device of any one of the preceding embodiments, wherein each two adjacent pillars in the same column further comprises a slit forming unit, defining the slit.

Embodiment 4. The device of any one of the preceding embodiments, wherein, when in operation, the device is configured to be applied with: a first electric field substantially in x direction and a second electric field substantially in y direction; and/or a first pressure substantially in x direction and a second pressure substantially in y direction.

Embodiment 5. The device of embodiment 4, wherein the first electric field is about 5-1,000 Vcm⁻¹, and the second electric field is about 5-1,000 Vcm⁻¹.

Embodiment 6. The device of embodiment 4, wherein the first electric field is about 40-250 Vcm 1, and the second electric field is about 60-300 Vcm⁻¹.

Embodiment 7. The device of any one of the preceding embodiments, wherein the slit depth is about 0.5-2.0 μm and the well depth is about 1-8 μm; and/or the slit length is about 0.5-2.0 μm and the well width is about 1-4 μm.

Embodiment 8. The device of any one of the preceding embodiments, wherein the slit depth is about 0.7-1.0 μm and the well depth is about 4 μm; and/or the slit length is about 0.7-1.0 μm and the well width is about 4 μm.

Embodiment 9. The device of any one of the preceding embodiments, wherein the sieving array is configured to be pre-filled with a buffer.

Embodiment 10. The device of any one of embodiment 9, wherein the buffer comprises tris-borate-EDTA (TBE) buffer, Tris-Acetate-EDTA (TAE) Buffer, Tris-Glycine (TG) Buffer, Phosphate Buffer Saline (PBS) Buffer, or combination thereof.

Embodiment 11. The device of any one of the preceding embodiments, wherein the buffer further comprises an agent that is able to suppress or stabilize electroosmotic flow (EOF).

Embodiment 12. The device of embodiment 11, wherein the agent comprises Performance Optimized Polymer-6 (POP-6), Performance Optimized Polymer-4 (POP-4), Performance Optimized Polymer-7 (POP-7), polyethylene oxide (PEO) or combination thereof.

Embodiment 13. The device of any one of the preceding embodiments, wherein the device is configured to fractionate the particles in the sample, and wherein the sample comprises one or more of: extracellular vesicles (EVs); proteins or protein aggregates; nucleic acids; liposomes; polymeric, lipid-based or inorganic nanoparticles; quantum dots; carbon nanotubes; metal particles; colloidal particles; nanowires; microplastics or nanoplastics; viruses; viral vectors; and combination thereof.

Embodiment 14. The device of any one of the preceding embodiments, wherein the particles have particle sizes ranged from about 30 nm to 1 μm.

Embodiment 15. The device of any one of the preceding embodiments, wherein the particles have particle sizes ranged from about 70 nm to 300 nm.

Embodiment 16. A microfluidic device for size fractionation, separation or purification of extracellular vesicles (EVs) in a sample, comprising: at least one sample inlet connected with an injection channel for sample loading; a plurality of buffer reservoirs; a sieving array; and a plurality of outlets connected with sample collection reservoirs for collecting sorted EVs, wherein the sample inlet, the at least one reservoir and the at least one particle collection reservoir are in fluid communication with the sieving array via one or more microchannels, respectively, wherein the sieving array comprises a substrate and a plurality of pillars, the plurality of pillars are spaced from one another and arranged in an array of a plurality of rows substantially in x direction and a plurality of columns substantially in y direction on the substrate, wherein each two adjacent columns define a well therebetween, thereby forming a plurality of wells in the sieving array,
wherein each two adjacent pillars in the same column further define a slit therebetween, thereby forming a plurality of slits in the sieving array,
wherein each well has a well depth and a well width, and each slit has a slit depth and a slit length;
and wherein the slit depth is configured to be smaller than the well depth; and/or the slit length is configured to be smaller than the well width, such that, when in operation, EVs are driven to pass through one or more of the plurality of slits and/or one or more of the plurality of wells based on at least particle size, thereby EVs are sorted and collected in the plurality of outlets.

Embodiment 17. The device of embodiment 16, wherein the device is configured to receive a first electric field substantially in x direction and a second electric field substantially in y direction, respectively, and wherein the first electric field is about 40-250 Vcm⁻¹, and the second electric field is about 60-300 Vcm⁻¹.

Embodiment 18. The device of any one of the embodiments 16-17, wherein the slit depth is about 0.5-2.0 μm, and the well depth is about 1-8 μm; and/or the slit length is about 0.5-2.0 μm and the well width is about 1-4 μm.

Embodiment 19. The device of any one of the embodiments 16-18, wherein the slit depth is about 0.5-1.0 μm and the well depth is about 4 μm; and/or the slit length is about 0.7-1.0 μm and the well width is about 4 μm.

Embodiment 20. A method for sorting particles in a sample, comprising the steps of: (a) providing a device as described in any one of the embodiments 1-19; (b) loading a sample to the sample inlet of the device; and (c) flowing the sample into the device, such that particles are sorted and collected in the at least one outlet.

Embodiment 21. The method of embodiment 20, the step (c) further comprises the step of: applying a first electric field substantially in x direction and a second electric field substantially in y direction; and/or applying a first pressure substantially in x direction and a second pressure substantially in y direction to drive the particles to migrate.

Embodiment 22. The method of embodiment 20 or 21, prior to the step (b), preparing the device by pre-filling a buffer to the sieving array, wherein the buffer comprises tris-borate-EDTA (TBE) buffer, Tris-Acetate-EDTA (TAE) Buffer, Tris-Glycine (TG) Buffer, Phosphate Buffer Saline (PBS) Buffer or combination thereof.

Embodiment 23. The method of embodiment 22, wherein the buffer further comprises an agent that is able to suppress or stabilize electroosmotic flow (EOF), wherein the agent comprises Performance Optimized Polymer-6 (POP-6), Performance Optimized Polymer-4 (POP-4), Performance Optimized Polymer-7 (POP-7), polyethylene oxide (PEO) or combination thereof.

Embodiment 24. A method for size fractionation, separation or purification of extracellular vesicles (EVs) in a sample, comprising the steps of: (a) providing a device as described in any one of the embodiments 16-19; (b) loading a sample to the sample inlet of the device; and (c) flowing the sample into the device, such that EVs are sorted and collected in the at least one outlet.

Embodiment 25. The method of embodiment 24, the step (c) further comprises the step of: applying a first electric field substantially in x direction and a second electric field substantially in y direction; and/or applying a first pressure substantially in x direction and a second pressure substantially in y direction to drive the particles to migrate.

Embodiment 26. The method of embodiment 24 or 25, prior to the step (b), preparing the device by pre-filling a buffer to the sieving array, wherein the buffer comprises tris-borate-EDTA (TBE) buffer, Tris-Acetate-EDTA (TAE) Buffer, Tris-Glycine (TG) Buffer, Phosphate Buffer Saline (PBS) Buffer or combination thereof.

Embodiment 27. The method of any one of the embodiments 24-26, the buffer further comprises an agent that is able to suppress or stabilize electroosmotic flow (EOF), wherein the agent comprises Performance Optimized Polymer-6 (POP-6), Performance Optimized Polymer-4 (POP-4), Performance Optimized Polymer-7 (POP-7), polyethylene oxide (PEO) or combination thereof.

All patents, patent applications, provisional applications, and publications referred to or cited herein are incorporated by reference in their entirety, including all figures and tables, to the extent they are not inconsistent with the explicit teachings of this specification.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A microfluidic device for sorting particles in a sample, comprising:
a sample inlet for sample loading;
at least one reservoir;
a sieving array; and
at least one outlet for collecting any sorted particles,
wherein the sample inlet, the at least one reservoir and the at least one particle collection reservoir are in fluid communication with the sieving array, respectively,
wherein the sieving array comprises a substrate and a plurality of pillars, the plurality of pillars are spaced from one another and arranged in an array of a plurality of rows substantially in x direction and a plurality of columns substantially in y direction,
wherein each two adjacent columns define a well therebetween, thereby forming a plurality of wells in the sieving array,
wherein each two adjacent pillars in the same column further define a slit therebetween, thereby forming a plurality of slits in the sieving array,
such that, when in operation, particles are driven to pass through one or more of the plurality of slits and/or one or more of the plurality of wells based on at least particle size, thereby particles are sorted and collected from the at least one outlet.

2. The device of claim 1, wherein each well has a well depth and a well width, and each slit has a slit depth and a slit length; and
wherein the slit depth is configured to be smaller than the well depth; and/or the slit length is configured to be smaller than the well width.

3. The device of claim 1, wherein each two adjacent pillars in the same column further comprises a slit forming unit, defining the slit.

4. The device of claim 1, wherein, when in operation, the device is configured to be applied with:
a first electric field substantially in x direction and a second electric field substantially in y direction; and/or
a first pressure substantially in x direction and a second pressure substantially in y direction.

5. The device of claim 4, wherein the first electric field is about 5-1,000 Vcm⁻¹, and the second electric field is about 5-1,000 Vcm⁻¹.

6. The device of claim 4, wherein the first electric field is about 40-250 Vcm⁻¹, and the second electric field is about 60-300 Vcm⁻¹.

7. The device of claim 2, wherein the slit depth is about 0.5-2.0 μm and the well depth is about 1-8 μm; and/or the slit length is about 0.5-2.0 μm and the well width is about 1-4 μm.

8. The device of claim 2, wherein the slit depth is about 0.7-1.0 μm and the well depth is about 4 μm; and/or the slit length is about 0.7-1.0 μm and the well width is about 4 μm.

9. The device of claim 1, wherein the sieving array is configured to be pre-filled with a buffer.

10. The device of claim 9, wherein the buffer comprises tris-borate-EDTA (TBE) buffer, Tris-Acetate-EDTA (TAE) Buffer, Tris-Glycine (TG) Buffer, Phosphate Buffer Saline (PBS) Buffer, or combination thereof.

11. The device of claim 9, wherein the buffer further comprises an agent that is able to suppress or stabilize electroosmotic flow (EOF).

12. The device of claim 11, wherein the agent comprises Performance Optimized Polymer-6 (POP-6), Performance Optimized Polymer-4 (POP-4), Performance Optimized Polymer-7 (POP-7), polyethylene oxide (PEO) or combination thereof.

13. The device of claim 1, wherein the device is configured to fractionate the particles in the sample, and wherein the sample comprises one or more of: extracellular vesicles (EVs); proteins or protein aggregates; nucleic acids; liposomes; polymeric, lipid-based or inorganic nanoparticles; quantum dots; carbon nanotubes; metal particles; colloidal particles; nanowires; microplastics or nanoplastics; viruses; viral vectors; and combination thereof.

14. The device of claim 1, wherein the particles have particle sizes ranged from about 30 nm to 1 μm.

15. The device of claim 1, wherein the particles have particle sizes ranged from about 70 nm to 300 nm.

16. A microfluidic device for size fractionation, separation or purification of extracellular vesicles (EVs) in a sample, comprising:
at least one sample inlet connected with an injection channel for sample loading;
a plurality of buffer reservoirs;
a sieving array; and
a plurality of outlets connected with sample collection reservoirs for collecting sorted EVs,
wherein the sample inlet, the at least one reservoir and the at least one particle collection reservoir are in fluid communication with the sieving array via one or more microchannels, respectively,
wherein the sieving array comprises a substrate and a plurality of pillars, the plurality of pillars are spaced from one another and arranged in an array of a plurality of rows substantially in x direction and a plurality of columns substantially in y direction on the substrate,
wherein each two adjacent columns define a well therebetween, thereby forming a plurality of wells in the sieving array,
wherein each two adjacent pillars in the same column further define a slit therebetween, thereby forming a plurality of slits in the sieving array,
wherein each well has a well depth and a well width, and each slit has a slit depth and a slit length; and
wherein the slit depth is configured to be smaller than the well depth; and/or the slit length is configured to be smaller than the well width,
such that, when in operation, EVs are driven to pass through one or more of the plurality of slits and/or one or more of the plurality of wells based on at least particle size, thereby EVs are sorted and collected in the plurality of outlets.

17. The device of claim 16, wherein the device is configured to receive a first electric field substantially in x direction and a second electric field substantially in y direction, respectively, and wherein the first electric field is about 40-250 Vcm⁻¹, and the second electric field is about 60-300 Vcm⁻¹.

18. The device of claim 16, wherein the slit depth is about 0.5-2.0 μm, and the well depth is about 1-8 μm; and/or the slit length is about 0.5-2.0 μm and the well width is about 1-4 μm.

19. The device of claim 16, wherein the slit depth is about 0.5-1.0 μm and the well depth is about 4 μm; and/or the slit length is about 0.7-1.0 μm and the well width is about 4 μm.

20. A method for sorting particles in a sample, comprising the steps of:
(a) providing a device as claimed in claim 1;
(b) loading a sample to the sample inlet of the device; and
(c) flowing the sample into the device, such that particles are sorted and collected in the at least one outlet.

21. The method of claim 20, the step (c) further comprises the step of: applying a first electric field substantially in x direction and a second electric field substantially in y direction; and/or
applying a first pressure substantially in x direction and a second pressure substantially in y direction to drive the particles to migrate.

22. The method of claim 20, prior to the step (b), preparing the device by pre-filling a buffer to the sieving array, wherein the buffer comprises tris-borate-EDTA (TBE) buffer, Tris-Acetate-EDTA (TAE) Buffer, Tris-Glycine (TG) Buffer, Phosphate Buffer Saline (PBS) Buffer or combination thereof.

23. The method of claim 22, wherein the buffer further comprises an agent that is able to suppress or stabilize electroosmotic flow (EOF), wherein the agent comprises Performance Optimized Polymer-6 (POP-6), Performance Optimized Polymer-4 (POP-4), Performance Optimized Polymer-7 (POP-7), polyethylene oxide (PEO) or combination thereof.

24. A method for size fractionation, separation or purification of extracellular vesicles (EVs) in a sample, comprising the steps of:
(a) providing a device as claimed in claim 16;
(b) loading a sample to the sample inlet of the device; and
(c) flowing the sample into the device, such that EVs are sorted and collected in the at least one outlet.

25. The method of claim 24, the step (c) further comprises the step of: applying a first electric field substantially in x direction and a second electric field substantially in y direction; and/or applying a first pressure substantially in x direction and a second pressure substantially in y direction to drive the particles to migrate.

26. The method of claim 24, prior to the step (b), preparing the device by pre-filling a buffer to the sieving array, wherein the buffer comprises tris-borate-EDTA (TBE) buffer, Tris-Acetate-EDTA (TAE) Buffer, Tris-Glycine (TG) Buffer, Phosphate Buffer Saline (PBS) Buffer or combination thereof.

27. The method of claim 26, wherein the buffer further comprises an agent that is able to suppress or stabilize electroosmotic flow (EOF), wherein the agent comprises Performance Optimized Polymer-6 (POP-6), Performance Optimized Polymer-4 (POP-4), Performance Optimized Polymer-7 (POP-7), polyethylene oxide (PEO) or combination thereof.
